(19) United States
(12) Patent Application Publication
(10) Pub. No.: US 2017/0121687 A1
(43) Pub. Date: May 4, 2017

(54) METHODS OF CELLULAR REPROGRAMMING

(71) Applicant: Agency for Science, Technology and Research, Singapore (SG)

(72) Inventors: Andrew Chwee Aun WAN, Singapore (SG); Jia Kai LIM, Singapore (SG); Vivian Yujing LIM, Singapore (SG); Nina MA, Singapore (SG); Siti Thaharah MOHAMED, Singapore (SG)

(21) Appl. No.: 15/306,045

(22) PCT Filed: Apr. 23, 2015
(86) PCT No.: PCT/SG2015/050082

§ 371 Date: Oct. 21, 2016

(30) Foreign Application Priority Data
Apr. 23, 2014 (SG) 10201401734X

(51) Int. Cl. C12N 5/09; A61K 35/36; A61K 35/13; C12N 5/077; A61K 35/33

(52) U.S. Cl. CPC C12N 5/0693; C12N 5/0656; A61K 35/33; A61K 35/13; A61K 35/36; C12N 2501/585

(57) ABSTRACT

The present invention relates to a method for reprogramming a first cell type to an intermediate cell of a second cell type comprising the step of contacting the first cell with a first agent to modulate an integrin profile in the first cell type to provide an intermediate cell of the second cell type. The present invention also relates to a reprogrammed cell obtained by the method of the invention, a kit for reprogramming a first cell type to a second cell type as well as methods for treating a patient in need of cell based therapy, tissue replacement and cancer therapy.

### CROSS-REFERENCE TO RELATED APPLICATIONS

This application claims the benefit of priority of Singapore application no. 10201401734X, filed 23 Apr. 2014, the contents of it being hereby incorporated by reference in its entirety for all purposes.

### FIELD OF THE INVENTION

The present invention relates to cellular reprogramming. Specifically, the present invention relates to integrin modulated cellular reprogramming.

### BACKGROUND OF THE INVENTION

Phenotypic stability is a hallmark of differentiated cells. This phenotypic stability is maintained by reciprocal interactions between the cell and its environment in a process known as dynamic reciprocity, where signals between the cellular microenvironment, such as the extracellular matrix (ECM), and the cell interact to influence and stabilize the cell phenotype within its niche environment.

The linkage between the cell and its microenvironment involves transmembrane cell adhesion proteins, of which the integrins form a superfamily. Integrins therefore play a critical role in cell adhesion to the ECM, which in turn, regulate cellular processes of adhesion, proliferation, migration and differentiation.

However, to date, there are few reports on reprogramming differentiated cells to stem-cell like cells, or of reprogramming differentiated cells to other differentiated cell types by manipulating the interaction of the cell with the ECM. There are also few reports on the role that integrins play in reprogramming the fate of differentiated cells.

Accordingly, there is a need to understand the interactions of the cell with the ECM in reprogramming cell fate and the role integrins play in this process.

### SUMMARY OF THE INVENTION

In one aspect, there is provided a method for reprogramming a first cell type to an intermediate cell of a second cell type, comprising the step of: contacting said cell with a first agent to modulate an integrin profile in the first cell type to provide said intermediate cell of said second cell type.

In another aspect, there is provided a reprogrammed cell obtained according to the method as disclosed herein.

In another aspect, there is provided a kit for reprogramming a first cell type to a second cell type, comprising one or more of the following components: (i) a composition comprising at least one first agent to effect reprogramming of a first cell type to an intermediate cell type; (ii) a composition comprising at least one second agent to effect reprogramming of an intermediate cell type to a second cell type; optionally comprising instructions for use.

In another aspect, there is provided a method for treating a patient in need of cell-based therapy or tissue replacement, comprising administering to said patient a reprogrammed cell obtained according to the method as disclosed herein.

In another aspect, there is provided a reprogrammed cell obtained according to the method as disclosed herein for use in treating a patient in need of cell-based therapy or tissue replacement, wherein said reprogrammed cell is to be administered to said patient.

In another aspect, there is provided a use of a reprogrammed cell obtained according to the method as disclosed herein in the manufacture of a medicament for treating a patient in need of cell-based therapy or tissue replacement, wherein said medicament is to be administered to said patient.

In another aspect, there is provided a method for treating a patient in need of cancer therapy, comprising delivering a bioactive to said patient using as a vehicle, a reprogrammed cell obtained according to the method as disclosed herein.

In another aspect, there is provided a reprogrammed cell obtained according to the method as disclosed herein as a vehicle for delivering a bioactive for use in treating a patient in need of cancer therapy.

In another aspect, there is provided a use of a reprogrammed cell obtained according to the method as disclosed herein as a vehicle for delivering a bioactive in the manufacture of a medicament for treating a patient in need of cancer therapy.

In another aspect, there is provided a method of determining the suitability of a cancer therapy for a cancer patient, comprising preparing a reprogrammed cell according to the method as disclosed herein, and administering to said reprogrammed cell one or more cancer therapy to assess efficacy of said cancer therapy on said reprogrammed cell.

In another aspect, there is provided a reprogrammed cell prepared according to the method as disclosed herein for use in determining the suitability of a cancer therapy for a cancer patient, wherein one or more cancer therapy is to be administered to said reprogrammed cell to assess efficacy of said cancer therapy on said reprogrammed cell.

### BRIEF DESCRIPTION OF THE DRAWINGS

The invention will be better understood with reference to the detailed description when considered in conjunction with the non-limiting examples and the accompanying drawings, in which:

FIG. 1. Timeline for induction by selective integrin inhibition and ligation

FIG. 2. Selective integrin inhibition leading to the intermediate phenotype. (A) The intermediate phenotype in the form of spherical cell clusters (SCC) at 3 days induction; Changes in (B) MET markers and (C) Stem cell markers, with respect to isotype control; (D): Western blot showing upregulation of expression for the integrins α2 and α6, and to a lesser extent, integrin α3 and CXCR4

FIG. 3. Confocal micrographs of DAPI-stained (A) induced IMR90 fibroblasts (the intermediate phenotype) in comparison to (B) control fibroblasts, showing differences in chromatin and nuclear structure. Blue: DAPI staining; Green: phalloidin staining;

corresponding panel below shows 3D reconstruction of confocal sections through nuclei.

FIG. 4. (A) SCCs were cultured with ECM overnight; media was changed to B27 containing FGF and EGF. (i) Appearance of cells before media change; Appearance of cells following media change, after (ii) 1 day; (iii) 3 days; (iv) 4 days; (B) SCCs cultured with ECM overnight; media was changed to DMEM without growth factors. Appearance of cells following media change, after (ii) 1 day; (iii) 3 days; (iv) 4 days; (C) Gene expression after 4 days of culture. (C): Integrins profile after 4 days in ECM (Geltrex/Laminin 332) culture, with and without growth factors (GFs). (D) Epithelial and mesenchymal markers after 4 days in ECM (Geltrex/Laminin 332) culture, with and without GFs. (E) Pluripotent and stemness markers after 4 days in ECM (Geltrex/Laminin 332) culture, with and without GFs.

FIG. 5. Phenotype of reprogrammed cells. Gene expression (A) and cell morphology (B) of (i) geltrex-induced, and (ii) laminin-induced cells (with growth factors). In (B), cells were sub-cultured for 6 days.

FIG. 6. Reprogramming to neuronal phenotype. (A,B) Fibroblasts induced on Geltrex in the presence of Rin5f conditioned media, stained for Beta III tubulin, After 24 h, the cells were replated on TCP and cultured with (A) DM EM, or (B) SKP medium. Inset: Nuclear staining with DAPI showing cells of neuronal morphology stained for Beta III tubulin; (C, D) Reprogrammed fibroblasts exhibiting neuronal morphology, at higher magnification.

FIG. 7. 3-antibody combination comprising the integrins αv, αvβ5 and αvβ6 applied on a glioma cell line, U251 for 2 days. (A) Morphologies of antibody treated cells and (B) isotype controls. (C) Changes in the expression of various stemness and (D) MET markers, normalized against the corresponding isotype controls.

FIG. 8. Immunoblot of glioma cell line, U251 treated for 2 days with a 3-antibody combination comprising the integrins αv, αvβ5 and αvβ6.

FIG. 9. Antibody treated cells cultured on laminin coated culture wells. (A) Antibody treated cells. (B) Isotype control. (C) Immunoblot of glioma cell line, U251 treated for 2-days with a 3-antibody combination comprising the integrins αv, αvβ5 and αvβ6, then replated onto Laminin 511 and cultured for a further 4 days.

FIG. 10. Overexpression of individual integrins (Integrins α2, α3, α6, β1 and β4) is associated with increased expression of epithelial and stemness markers and decreased expression of mesenchymal markers. The changes are more pronounced when cells are cultured on (B) 500 nm silica nanotopographical scaffold, than on (A) glass coverslips.

FIG. 11. (A) Gene expression profile of human dermal fibroblast (HDF) 3 days after induction. (B) Gene expression profile of dermal papilla cells (DP) 2 days after induction.

FIG. 12. (A) Integrin expression profile. (B) Epithelial and mesenchymal markers. (C) Pluripotent and stemness markers of IMR90 4 days after induction, with exposure to ECM (Laminin 511 and Laminin 521) at Day 3. (D) Schematic diagram of reprogramming timeline.

FIG. 13. (A) Integrin expression profile. (B) Epithelial and mesenchymal markers. (C) Pluripotent and stemness markers of IMR90 10 days after induction, with exposure to laminin 521 at Day 3 and KGF and Day 4, respectively. (D) Schematic diagram of reprogramming timeline.

FIG. 14. (A) Integrin expression profile. (B) Epithelial and mesenchymal markers. (C) Pluripotent and stemness markers of IMR90 4 days after induction, with exposure to laminin 521 (KGF) and Day 1. (D) Schematic diagram of reprogramming timeline.

FIG. 15. (A) Integrin expression profile. (B) Epithelial and mesenchymal markers. (C) Pluripotent and stemness markers of IMR90 8 days after induction, with exposure to laminin 511 (+/−HGF) at Day 2. (D) Schematic diagram of reprogramming timeline.

FIG. 16. (A) Integrin expression profile. (B) Epithelial and mesenchymal markers. (C) Pluripotent and stemness markers of sarcoma lines 3 days after induction.

FIG. 17. (A) Integrin expression profile. (B) Epithelial and mesenchymal markers. (C) Pluripotent and stemness markers of hMSCs 3 days after induction. (D) Schematic diagram of reprogramming timeline.

### DEFINITIONS

The following words and terms used herein shall have the meaning indicated:

The terms “reprogramming”, “reprogram” and grammatical variants thereof as used herein refer to altering or reversing the differentiation status of a cell that is either undifferentiated, partially or terminally differentiated. As used herein, reprogramming may be partial or complete conversion of the differentiation status of a cell.

As used herein, the term “modulating”, “modulate” and grammatical variants thereof in reference to integrins refers to altering the gene expression and/or protein activity of one or more integrins. Integrin gene expression and/or protein activity may be modulated by one or more of integrin inhibitors, antagonists, activators, agonists, selective expression of integrins, overexpression of integrins, silencing expression of integrins, reducing expression levels of integrins, expression of additional integrins, selective inhibition of integrins, selective integrin ligation and inhibition of selected micro RNA (miRNA). Integrin gene expression and/or activity may also be modulated by altering the expression, levels and type of integrin ligands in the cellular microenvironment.

As used herein, the term “integrins” refer to one or more members of the integrin superfamily of cell adhesion receptors. The term “selection of integrins” refers to a panel of one or more integrins whose expression or overexpression primes a cell for reprogramming to a cell type.

As used herein, the term “integrin profile” refers to the gene expression and gene expression levels of one or more integrins in a cell. The term “integrin profile” may also refer to the activity of one or more integrins in a cell.

As used herein, the term “integrin ligand” refers to any molecule or agent that is capable of binding to one or more integrin subunits or integrin molecules or one or more integrin genes in a cell. Integrin ligands include but are not limited to RGD ligands, LDV ligands, anti-integrin antibodies or fragments thereof, extracellular matrix proteins or cell surface adhesion proteins.

As used herein, the term “nucleic acid molecule” refers to any single or double-stranded RNA or DNA molecule, such as mRNA, cDNA, genomic DNA, plasmid DNA, and xeno DNA.

As used herein, the term “intermediate cell” in reference to reprogramming a first cell type to a second cell type refers to a cell that displays phenotypic and/or genotypic characteristics of both the first and second cell types and/or additional characteristics not present in the first or second cell types. Phenotypic characteristic include but are not limited to proliferation rate, morphology, growth rate, developmental and biochemical properties. For example, an intermediate cell may be identified based on the expression of stem cell markers. In another example, an intermediate cell may be identified based on the expression of markers associated with mesenchyme to epithelial transition (MET). It would be generally understood that an intermediate cell exhibits a specific gene expression trend relative to the first cell type. For example, an intermediate cell may upregulate the expression of at least three markers of the second cell type and/or downregulate the expression of at least three markers of the first cell type. In one example, an intermediate cell may upregulate at least three epithelial markers and/or downregulate at least three mesenchymal markers compared to the first cell type. In another example, an intermediate may upregulate at least 3 stem cell markers and downregulate at least three epithelial markers compared to the first cell type. In another example, intermediate cells may display an intermediate phenotype such as spherical cell clusters. In yet another example, an intermediate cell may upregulate the expression of at least one integrin. For example, an intermediate cell may upregulate integrin β4 and integrin β6. An intermediate cell may also upregulate the expression of one or more of integrin α2, α3, α6, αv, β4 or β6. It will be generally understood that upregulation or downregulation of expression may be at the gene and/or protein level. It will also be generally understood that upregulation or downregulation is relative to either the first cell type and/or the second cell type.

As used herein, the term “inhibitor” in reference to integrins refers to an agent that interferes with the ability of one or more integrins to interact with its substrate. An inhibitor may be an antibody, an alternative substrate or an antagonist. An inhibitor or integrin may function by competitively binding to a substrate, altering the binding affinity of an integrin to a substrate or inducing conformational changes in an integrin molecule thereby altering the interaction of one or more integrins with its substrate.

The term “antibody” is used herein in the broadest sense to refer to molecules with an immunoglobulin-like domain and includes monoclonal, recombinant, polyclonal, chimeric, humanised, bispecific and heteroconjugate antibodies; a single variable domain, a domain antibody, antigen binding fragments, immunologically effective fragments, single chain Fv, diabodies, Tandabs™, etc (for a summary of alternative “antibody” formats see Holliger and Hudson, Nature Biotechnology, 2005, Vol 23, No. 9, 1126-1136). An antigen binding fragment or an immunologically effective fragment may comprise partial heavy or light chain variable sequences. Fragments are at least 5, 6, 8 or 10 amino acids in length. Alternatively the fragments are at least 15, at least 20, at least 50, at least 75, or at least 100 amino acids in length.

As used herein, the term “stem cell” refers to an undifferentiated or partially differentiated cell. A stem cell may be multipotent or pluripotent and may be isolated from a terminally differentiated tissue or organ (adult stem cells), derived from embryonic tissue (embryonic stem cells) or may be derived from a somatic cell or terminally differentiated cell by induction (induced pluripotent stem cells). A person of skill in the art would readily understand that a stem cell possesses specific genotypic and phenotypic characteristics. For example, a stem cell would express genes including but not limited to OCT4A, NANOG, SOX2, LIN28A, Nestin, CXCR4, TERT, TP63 and CSPG4.

Consequently, the term “stem cell-like cell” refers to a cell that has characteristics that resemble those of a stem cell. It will be understood that the resemblance of a stem cell-like cell to a stem cell may be partial or complete.

As used herein, the term “epithelial cell” refers to cells of the epithelia and include but are not limited to simple squamous epithelia, simple cuboidal epithelia, simple columnar epithelia, ciliated columnar epithelia, glandular epithelia, stratified epithelia and pseudostratified epithelia. It will be readily understood that epithelial cells possess specific genotypic and/or phenotypic characteristics.

Consequently, the term “epithelial-like cell” refers to cell that has characteristics that resemble those of one or more epithelial cells. It will be understood that the resemblance of an epithelial-like cell to an epithelial cell may be partial or complete.

As used herein, the term “mesenchymal cell” refers to cells of the mesenchyme. Consequently, the term “mesenchymal-like cell” refers to a cell that has genotypic and/or phenotypic characteristics that resemble one or more mesenchymal cells. It will be understood that the resemblance of a mesenchymal-like cell to mesenchyme cell may be partial or complete.

A person of skill in the art would easily recognize a cell having epithelial or epithelial-like, mesenchyme, or mesenchymal-like characteristics based on the characteristics of the cell. For example, the expression of specific genes, morphology and proliferation. Examples of epithelial and mesenchymal genes or markers include but are not limited to CDH1, DAG1, EPCAM, KRT18, LAMAS, LAMBS, LAMC2, PRRX1, ZEB1, CDH2, VIM, FN1, Snail, Slug and MMP9.

As used herein the term, “modifying” a cell type refers to altering the phenotypic and/or genotypic characteristics of a cell.

As used herein, the term “ligation” in reference to integrins refers to the binding of one or more agents or substrates to one or more integrins to effect integrin activity.

As used herein, the term “extracellular matrix” (ECM) refers to the network of proteins and polysaccharides secreted by cells. The ECM provides structural support and influences physiological, biochemical and developmental processes of cells in its microenvironment.

As used herein, the term “agent” refers to any type of molecule for example, a polynucleotide, a peptide, a peptidomimetic, peptoid, chemical compounds such as small molecules, organic molecules or the like.

As used herein, the term “therapy”, “treatment” or grammatical variants thereof refers to the alleviation of symptoms associated with a condition. Consequently, the term “cancer therapy” as used herein refers to the alleviation of symptoms associated with cancer.

As used herein, the term “cell-based therapy”, “cell therapy” or “cytotherapy” refers to therapy involving the use of cellular material. Cellular material may include intact cells or fragments of cells, allogenic cells derived from a donor or cells derived from the patient. Cellular material may also include xenogenic cells.

As used herein, the term “tissue replacement” refers to the process of replacing or repairing whole or partial tissue or organs in a patient. Replacement tissue or organs are synthesized two-dimensionally or three-dimensionally on scaffolds prior to administration to a patient.

As used herein, the term “scaffold” refers to a structure that provides support for cell attachment. A scaffold may be a 3-dimensional structure that mimics tissue such as the extracellular matrix or a cellular support that mimics a basement membrane. An example of a scaffold may be a three-dimensional support coated with extracellular matrix proteins. Another example of a scaffold may be a filter such as a Transwell membrane. Yet another example of a scaffold is a nanotopographical scaffold. A scaffold may be uncoated or coated with naturally occurring or synthetic molecules or compound. For example, a scaffold may be coated with collagen, glycosaminoglycans, peptides, peptidomimetics or poly-L-lysine.

As used herein, the term “bioactive” refers to an agent having biological activity.

As used herein, the term “growth factor” refers to a substance that is capable of stimulating cellular growth, proliferation or cellular differentiation. Growth factors typically act as signaling molecules between cells.

As used herein, the term “vehicle” refers to a means to deliver a bioactive to a patient.

As used herein, the term “suitability of a cancer therapy” refers to whether a patient is a candidate for a type of cancer therapy.

As used herein, “composition” or “pharmaceutical composition” refers to a mixture of one or more of the compounds described herein, or physiologically/pharmaceutically acceptable salts or prodrugs thereof, with other chemical components, such as physiologically/pharmaceutically acceptable carriers and excipients. The purpose of a pharmaceutical composition is to facilitate administration of a compound to an organism.

Compositions of the present invention may be manufactured by processes well known in the art, e. g., by means of conventional mixing, dissolving, granulating, dragee making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

As used herein, the terms “administration” or “administer” refer to the delivery of a modified collagen molecule or a pharmaceutically acceptable salt thereof or of a pharmaceutical composition containing modified collagen molecule or a pharmaceutically acceptable salt thereof of this invention to an organism for the purpose of wound healing, drug delivery and therapy.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. Alternatively, one may administer the collagen molecule in a local rather than systemic manner, for example, via injection of the compound directly into a tissue.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Disclosure of Optional Embodiments

Exemplary, non-limiting embodiments of a method for reprogramming a somatic cell to a stem cell-like phenotype, will now be disclosed:

The method for reprogramming a first cell type to an intermediate cell of a second cell type, comprising the step of: contacting said cell with a first agent to modulate an integrin profile in the first cell type to provide said intermediate cell of said second cell type.

In one embodiment, a cell is partially reprogrammed from a differentiated state to a less-differentiated state. In another embodiment, a cell is partially reprogrammed from an undifferentiated state to a partially differentiated state. In yet another embodiment, conversion of a cell is complete, for example, a terminally differentiated cell is converted into an undifferentiated cell or vice versa. In another embodiment, a terminally differentiated cell of one cell lineage is reprogrammed into a differentiated cell of another lineage. It will be understood that reprogramming may be unidirectional, from one cell type to another cell type, or bidirectional, from one cell type to another cell type and vice versa. Cellular reprogramming may take place in vitro, in vivo or ex vivo.

In one embodiment, the first agent is at least one integrin ligand. In one embodiment, contacting a cell with at least one integrin ligand may involve contacting a cell to one or more extracellular matrices or matrix proteins. Examples of extracellular matrices or matrix proteins include but are not limited to Geltrex®, laminin, fibronectin, gelatin, collagen I, collagen IV, AlgiMatrix™, Matrigel®, CTS™ CELLstart™, ornithine, vitronectin, entactin, ostepontin, osteonectin, tenascin C, ECM mimetics, or combinations thereof. In one embodiment, the extracellular matrix protein is one or more laminin isoforms. In a preferred embodiment, the extracellular matrix is Geltrex®, laminin 511, laminin 521 or laminin 332. In another embodiment, the extracellular matrix is derived from basement membrane. In yet another embodiment, the extracellular matrix is laminin 111, laminin 211, laminin 411 or laminin 421.

In one embodiment, contacting a cell with at least one integrin ligand may also involve contacting a cell type with one or more integrin inhibitors, integrin antagonists, peptides, cyclic peptides, disintegrins, peptidomimetics and small molecule antagonists. In one embodiment, the integrin inhibitor is an anti-integrin antibody or fragment thereof. Examples of an anti-integrin antibody or fragment thereof include but are not limited to anti-integrin αV, anti-integrin α5, anti-integrin beta 3, anti-integrin αV/β5, anti-integrin αVβ6. It will be understood to a person of skill in the art that antibodies directed to different clones may be used. For example, anti-integrin αV [272-17E6], anti-integrin αV (Clone JBS5), anti-integrin α5 (Clone H96), anti-integrin α5 [P1D6], anti-integrin beta 3 [25E11], anti-integrin αV/β5 (P1F6) L, anti-integrin αV/β6 10D5, anti-integrin β6 (Clone H-110).

In one embodiment, an integrin inhibitor binds to one or more integrin subunit selected from the group consisting of α1, α2, α3, α4, α5, α6, α7, α8, α9, α10, α11, αv, αD, αL, αM, αX, αE, αIIb, β1, β2, β3, β4, β5, β6, β7 and β8 and combinations thereof.

In one embodiment, the integrin inhibitor binds to one or more of αv, α5, β1, β3, αvβ5, αvβ6, β5, αvβ3 and β6.

In the method disclosed herein, contacting a cell with one or more integrin ligands alters gene expression to transition said first cell type to said intermediate cell type. In another embodiment, contacting a cell with one or more integrin ligands selects for a selection of integrins.

In one embodiment, an integrin ligand is contacted with a first cell type for a period sufficient to alter gene expression. For example, an integrin ligand is contacted with a first cell type for a period of about 1 minute, about 3 minutes, about 5 minutes, about 10 minutes, about 15 minutes, about 30 minutes, about 60 minutes, about 90 minutes, about 2 hours, about 24 hours, about 2 days, about 3 days and about 5 days. In one embodiment, the duration of contacting a first cell type with an integrin ligand is about 24 hours. In another embodiment, the duration of contacting a first cell type with an integrin ligand is about 2 days.

In another embodiment, the first agent that modulates an integrin profile in the first cell type is a nucleic acid molecule, wherein contacting said cell with said nucleic acid molecule alters the expression of at least one integrin gene, thereby modulating the integrin profile in said cell.

One example of a nucleic acid molecule is an expression vector. An expression vector may comprise one or more genes, each gene operably linked to a promoter. In another example, an expression vector may comprise one or more genes, operably linked to one promoter. Genes may be linked via linking sequences including but not limited to 2A and internal ribosome entry site (IRES). It will be generally understood that expression vectors may contain one or more selection markers. Expression vectors may be integrating or non-integrating vectors. An example of an expression vector is a viral expression vector. Examples of viral vectors include but are not limited to lentiviral expression vectors or plasmids, retroviral vectors, adeno-associated viral (AAV) vectors, baculoviral vectors and Sendai virus vectors. In one embodiment, the viral vector is a lentiviral vector.

In one example, a nucleic acid molecule may be an expression vector comprising at least one integrin gene operably linked to a promoter. In one example, a nucleic acid molecule is an expression vector that comprises at least one integrin gene operably linked to a promoter to overexpress said integrin gene in said cell.

In one example, the expression vector comprises at least one of integrins α2, α3, α6, β1 and β4 alone or in combination.

Other examples of a nucleic acid molecule include but are not limited to oligonucleotides such as interfering ribonucleic acids (iRNA). Examples of iRNA include but are not limited to small interfering ribonucleic acids (siRNA), micro RNA (miRNA), short hairpin RNA (shRNA).

It will be generally understood that “siRNA” refers to small interfering ribonucleic acids (RNA) or RNA analogs comprising between about 10 to 50 nucleotides (or nucleotide analogs) capable of directing or mediating the RNA interference pathway. These molecules can vary in length and can contain varying degrees of complementarity to their target messenger RNA (mRNA). The term “siRNA” includes duplexes of two separate strands, i.e. double stranded RNA, as well as single strands that can form hairpin structures comprising of a duplex region.

It will also be generally understood that the term “shRNA”, as used herein, refers to a unimolecular RNA that is capable of performing RNAi and that has a passenger strand, a loop and a guide strand. The passenger and guide strand may be substantially complementary to each other. The term “shRNA” may also include nucleic acids that contain moieties other than ribonucleotide moieties, including, but not limited to, modified nucleotides, modified internucleotide linkages, non-nucleotides, deoxynucleotides, and analogs of the nucleotides mentioned thereof.

It will be generally understood that miRNA is generally a single stranded molecule that averages about 20 nucleic acids.

Nucleic acid molecules may be contacted with a cell. Contacting a nucleic acid molecule with a cell may include methods generally known in the art. For example, methods including but not limited to chemical transfection, electroporation, heat shock, viral infection or microinjection.

In one example, contacting a first cell type with a nucleic acid molecule alters the expression of at least one integrin gene, wherein the at least one integrin gene is overexpressed. For example, one or more of the integrins α2, α3, α6, β1 and β4 are overexpressed.

In one example, contacting a first cell type with a nucleic acid molecule alters the expression of at least one integrin gene, wherein the at least one integrin gene is selectively expressed. In another example, one or more integrins may be silenced in a cell type using a nucleic acid molecule. In yet another example, one or more integrin expression levels may be reduced using a nucleic acid molecule. Integrin overexpression, selective expression of integrin, integrin gene silencing and/or reduction in integrin expression level may be tested using PCR, for example, semi-quantitative or quantitative PCR.

In the method disclosed herein, the method further comprises the step of detecting the expression of markers indicative of the intermediate cell type to determine that said first cell type has transitioned to said intermediate cell type. It will be generally understood that detecting the expression of a marker includes testing for the presence or absence of a marker or measuring the level of expression or change in level of expression of a marker. For example, the method further comprises the step of detecting the expression of one or more stem cell markers and/or mesenchyme to epithelial transition (MET) associated markers to determine that said first cell type has transitioned to said intermediate cell type. Examples of markers that may be used to determine that the first cell type has transitioned to an intermediate cell type include but are not limited to OCT4A, NANOG, SOX2, LIN28A, Nestin, CXCR4, TERT, TP63, CSPG4, TGF B1, FGF2, H1F1A, CDH1, DAG1, EPCAM, KRT18, CDH2, VIM, FN1, ZEB1 and PRRX1.

In another embodiment, the method further comprises the step of detecting the downregulation of markers associated with the first cell type to determine that said first cell type has transitioned to said intermediate cell type. For example, the method comprises the step of detecting downregulation of one or more epithelial markers to determine that said first cell type has transitioned to said intermediate cell type. Examples of epithelial markers include but are not limited to CDH1, DAG1, EPCAM and KRT18.

In one embodiment, the method further comprises the step of detecting the upregulation or downregulation of one or more integrin genes. For example, the upregulation of integrin beta 4 and integrin beta 6 relative to the first cell type is indicative of an intermediate cell. In another example, the upregulation of one or more of integrin α2, α3, α6, αv, β4 or β6 relative to the first cell type may be indicative of an intermediate cell. In another example, the upregulation of integrin beta 4 and integrin beta 6 relative to the second cell type is indicative of an intermediate cell. In yet another example, the upregulation of one or more of integrin α2, α3, α6, αv, β4 or β6 relative to the second cell type may be indicative of an intermediate cell.

In one embodiment, detecting the expression of pluripotent markers, epithelial markers, MET markers and/or integrin expression is by polymerase chain reaction (PCR). PCR may be real-time quantitative PCT or semi-quantitative PCR. Detecting the expression of stem cell markers may also be by immunohistochemistry, immunofluorescence, flow cytometry, DNA sequencing or microarray.

In one embodiment, the method provided herein further comprises contacting the intermediate cell type with a composition comprising a second agent to effect reprogramming of the intermediate cell type to the second cell type. For example, the agent may be a ligand that binds to one or more of the integrin subunits α1, α2, α3, α4, α5, α6, α7, α8, α9, α10, α11, αv, αD, αL, αM, αX, αE, αIIb, β1, β2, β3, β4, β5, β6, β7 and β8. In one embodiment, the second agent ligates one or more of the integrin subunits α2, α3, α6, αv, α5, β1, β3, β4, β5, β6 and/or one or more of the combination of subunits αvβ3, αvβ5, αvβ6, α6β1, α6β4, α2β1, α3β1 and α5β1.

In one embodiment, the second agent comprises one or more ligands that binds to one or more integrin subunits and/or combinations thereof to cause ligation of said integrin.

Examples of an agent that effects reprogramming of the intermediate cell type to the second cell type include but are not limited to components of the extracellular matrix and basement membrane. In one embodiment, contacting a cell with at least one integrin ligand may involve contacting a cell with one or more extracellular matrices or matrix proteins, for example, Geltrex®, laminin, fibronectin, gelatin, collagen I, collagen IV, AlgiMatrix™, Matrigel®, CTS™, CELLstart™, ornithine, vitronectin, entactin, osteopontin, osteonectin, tenascin C, ECM mimetics, or combinations thereof. In one embodiment, the extracellular matrix protein is one or more laminin isoforms. In another embodiment, the extracellular matrix is Geltrex®, laminin 511, laminin 521 or laminin 332. In another embodiment, the extracellular matrix is derived from basement membrane. In yet another embodiment, the extracellular matrix is laminin 111, laminin 211, laminin 411 or laminin 421.

In one embodiment, the one or more extracellular matrix components are derived from basement membrane.

In one embodiment, the agent that effects reprogramming of the intermediate cell type to the second cell type is contacted with the intermediate cell type for a duration sufficient to effect reprogramming to the second cell type.

For example, the duration of contact is at least about 12 hours, about 18 hours, about 24 hours, about 30 hours, about 36 hours, about 48 hours, about 2.5 days, about 3 days, about 3.5 days, about 4 days, about 4.5 days, about 5 days, about 5.5 days, about 6 days, about 6.5 days or about 7 days.

In the method disclosed herein, reprogramming of a first cell type to a second cell type provides a cell type that is intermediate between the first and second cell types. An intermediate cell type may have phenotypic and/or genotypic characteristic of the first and second cell types. For example, the intermediate cell type may have increased stem-cell like characteristics relative to the first cell type. In another example, the intermediate cell type may have increased epithelial like characteristics relative to the first cell type. An intermediate cell type may also have characteristics that are not observed in either the first or second cell types. It will be generally understood that increased stem-cell likes characteristics in an intermediate cell type relative to another cell type means that the intermediate cell type has more stem cell characteristics compared to another cell type. For example, the intermediate cell type may express more stem cell markers, higher levels of stem cell markers, or tend to form cell clusters. Similarly a cell that has increased epithelial characteristics compared to another cell would be understood to display more epithelial genotypic or phenotypic characteristics than said other cell.

Examples of cell types include but are not limited to mesenchymal cells, epithelial cells, multipotent cells, pluripotent cells, cancer stem cells and/or cells with partial characteristics of the above cell types. In one embodiment, the first or second cell type is a fibroblast cell, such as fetal lung fibroblast, IMR90 fibroblast, dermal fibroblast, adult dermal fibroblast. In another embodiment, the first or second cell type is a mesenchymal cell such as a human mesenchymal stem cell, follicle dermal papilla cell. In another embodiment, the first cell type is a cancer cell, for example, a primary cancer cell, a cancer stem cell or a cancer cell line. Examples of cancers include but are not limited to liposarcoma, fibrosarcoma, synovialsarcoma, rhabdomyosarcoma and melanoma.

In another embodiment, the first or second cell type is a stem cell-like cell, or a stem cell. In another embodiment, the first or second cell type is a neuronal cell, astrocyte or melanocyte. In yet another embodiment, the first cell is a stem cell or stem cell-like cell and the second cell type is an epithelial or epithelial-like cell, neuronal cells or cancer stem cells.

In another embodiment, the first or second cell type is a cell with stem cell-like and/or epithelial characteristics.

In one embodiment, a fibroblast may be reprogrammed to a neural cell.

In one embodiment, a fibroblast may be reprogrammed to a different somatic cell.

The present invention also provides a method for reprogramming a first cell type to a second cell type, comprising the step of:

(b) contacting a selection of integrins that is present and/or that has been induced to be expressed on the first cell type and that is associated with the reprogramming to the second cell type, with a composition comprising an agent that is capable of effecting the reprogramming to the second cell type.

In one embodiment, the selection of integrins may comprise one or more of α2, α3, α6, αv, β4 and β6 integrins.

In another embodiment, the method disclosed herein further comprises providing said first cell type that has been contacted with said first agent to provide said intermediate cell of said second cell type and said second agent to effect the reprogramming of said intermediate cell type to the second cell type with an additional agent that is capable of effecting further reprogramming and/or modification of the growth and/or proliferative characteristics of the first cell type.

Examples of the additional agent include but are not limited to a growth factor, conditioned medium, or supplemented cell culture medium. In one embodiment, the growth factor may be one or more of fibroblast growth factor 2 (FGF2), epidermal growth factor (EGF), hepatocyte growth factor (HGF) and keratinocyte growth factor (KGF). In another embodiment, the conditioned medium is conditioned from a cell of the second cell type. In yet another embodiment, the conditioned medium is conditioned from a cell type different to the first and second cell types. In one example, the conditioned medium is conditioned from a rat insulinoma (Rin5f) cell line. In one embodiment, the cell culture medium may be supplemented with B-27® supplement (comprising BSA, transferrin, insulin, progesterone, putrescine, sodium selenite, biotin, 1-carnitine, corticosterone, ethanolamine, d(+)-galactose, glutathione (reduced), linolenic acid, linoleic acid, retinyl acetate, selenium, t3 (triodo-1-thyronine), dl-α-tocopherol (vitamine e), dl-α-tocopherol acetate, catalase, superoxide dismutase). In another embodiment, the cell culture medium is TeSR™2 (comprising DMEM/F12 (liquid), L-ascorbic acid, selenium, transferrin, NaHCO3, glutathione, L-glutamine, defined lipids, thiamine, β-mercaptoethanol, albumin, insulin, FGF2, TGFβ1, pipecolic acid, LiCl, GABA).

In one embodiment, the duration of contact of the additional agent is for a period sufficient to effect further reprogramming to the second cell type and/or modification of the growth and/or proliferative characteristics of the cell.

In one embodiment, the duration of contact with the additional agent is at least about 1 day, about 2 days, about 3 days, about 4 days, about 5 days, about 6 day and about 7 days.

Cells reprogrammed by the methods as disclosed herein may be cultured on scaffolds. Scaffolds may be derived from natural sources or may be synthetic and may be ceramics, synthetic polymers or natural polymers. Scaffolds may also be composites comprised of different types of biomaterials. Examples of scaffold include but are not limited to polystyrene, poly-L-lactic acid, polyglycolic acid, collagen and collagen composites.

In one embodiment, a scaffold is a nanotopographical scaffold. In another embodiment, a scaffold is a nanotopographical scaffold comprising arrays of nanoparticles such as silica. For example, a nanotopographical scaffold may comprise of uniform arrays of silica nanoparticles of one or more sizes, for example two sizes. Nanoparticles may be about 1 nm, 5 nm, 10 nm, 15 nm, 20 nm, 25 nm, 30 nm, 35 nm, 40 nm, 45 nm, 50 nm, 55 nm, 60 nm, 65 nm, 70 nm, 80 nm, 85 nm, 90 nm, 95 nm, 100 nm, 110 nm, 120 nm, 130 nm, 140 nm, 150 nm, 160 nm, 170 nm, 180 nm. 190 nm, 200 nm, 210 nm, 220 nm, 230 nm, 240 nm, 250 nm, 260 nm, 270 nm, 280 nm, 290 nm, 300 nm, 310 nm 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm 480 nm, 490 nm, 500 nm, 550 nm, 600 nm, 650 nm, 700 nm, 750 nm, 800 nm, 850 nm, 900 nm, 950 nm or 1000 nm in diameter. Examples of suitable sizes of nanoparticles include but are not limited to 66±6 nm and 414±37 nm.

It will be generally understood that the first, second or additional agent may be used sequentially or simultaneously. For example, the first, second and additional agent may be added to a cell simultaneously. In another example, the first agent may be added, followed by the second agent, followed by the additional agent. In another example, the first and second agents may be added simultaneously, following by the addition of the additional agent. In another example, the first agent may be added, followed by the addition of the second and additional agents simultaneously.

It will also be generally understood that when one or more of the agents are used sequentially, the preceding agent(s) may be removed prior to the addition of the subsequent agent. It will also be generally understood that when one or more of the agents are used sequentially, the preceding agent(s) may not be removed prior to the addition of the subsequent agent.

For example, the first agent may be added to a first cell type to reprogram said first cell type into an intermediate cell type. Subsequently, the first agent may be removed and the second agent added. In another example, the first agent may be added to a first cell type to reprogram said first cell type into an intermediate cell type. Subsequently, the second agent may be added without removal of the first agent. In yet another example, the first agent may be added to effect reprogramming of a first cell type to an intermediate cell type, followed by the addition of the second and additional agent, without prior removal of the first agent. In yet another example, the first agent may be added to effect reprogramming of a first cell type to an intermediate cell type, followed by the addition of the second agent, without prior removal of the first agent, followed by the addition of the additional agent without prior removal of the first or second agents.

The methods disclosed herein may be conducted in vivo, in vitro or ex vivo.

Also provided herein is a reprogrammed cell obtained according to the methods disclosed herein.

Also provided herein is a kit for reprogramming a first cell type to a second cell type, comprising one or more of the following components: (i) a composition comprising at least one first agent to effect reprogramming of a first cell type to an intermediate cell type; (ii) a composition comprising at least one second agent to effect reprogramming of an intermediate cell type to a second cell type, optionally comprising instructions for use.

In one embodiment, the kit further comprises (a) a growth factor selected from the group consisting of fibroblast growth factor 2 (FGF2), epidermal growth factor (EGF), hepatocyte growth factor (HGF) and keratinocyte growth factor (KGF); and/or (b) a conditioned medium such as a conditioned medium from a rat insulinoma (Rin5f) cell line; and/or (c) a medium selected from B-27® supplement (comprising BSA, transferrin, insulin, progesterone, putrescine, sodium selenite, biotin, 1-carnitine, corticosterone, ethanolamine, d(+)-galactose, glutathione (reduced), linolenic acid, linoleic acid, retinyl acetate, selenium, t3 (triodo-1-thyronine), dl-α-tocopherol (vitamin e), dl-α-tocopherol acetate, catalase, superoxide dismutase) and TeSR™2 (comprising DMEM/F12 (liquid), L-ascorbic Acid, selenium, transferrin, NaHCO3, glutathione, L-glutamine, defined lipids, thiamine, β-mercaptoethanol, albumin, insulin, FGF2, TGFβ1, pipecolic acid, LiCl, GABA); and a scaffold for supporting growth of the cells being reprogrammed, wherein optionally, said scaffold comprises a nanotopographical scaffold, and wherein optionally said nanotopographical scaffold comprises uniform arrays of silica nanoparticles.

Also provided herein is a method for treating a patient in need of cell-based therapy or tissue replacement, comprising administering to said patient a reprogrammed cell obtained according to the method disclosed herein.

Also provided herein is a method for treating a patient in need of cancer therapy, comprising delivering a bioactive to said patient using as a vehicle, a reprogrammed cell obtained according to the method disclosed herein.

Also provided herein is a method of determining the suitability of a cancer therapy for a cancer patient, comprising preparing a reprogrammed cell (e.g. a cancer stem cell) according to the method as disclosed herein, and administering to said reprogrammed cell one or more cancer therapy to assess efficacy of said cancer therapy on said reprogrammed cell.

It will be understood that a reprogrammed cell may be derived from a donor or from the patient.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

Non-limiting examples of the invention, including the best mode, and a comparative example will be further described in greater detail by reference to specific Examples, which should not be construed as in any way limiting the scope of the invention.

Material and Methods

Cell Culture and Reprogramming to Stem Cell Phenotypes

Cells used in the study are IMR90 fibroblasts from American Type Culture Collection (Manassas, Va.), adult human dermal fibroblasts HDFa (Life Technologies, USA), follicle dermal papilla cells (Promocell, USA) and glioma cell line, U251. IMR90 cells were maintained in Dulbecco's modified Eagle's medium supplemented with 10% fetal bovine serum, glutamax and penicillin-streptomycin. HDFa was maintained in fibroblast basal media supplemented with the fibroblast growth kit-low serum from American Type Culture Collection (Manassas, Va.). Follicle dermal papilla cells were maintained in follicle dermal papilla cell growth medium (Promocell, USA). All cells were incubated in a 37° C. incubator with 5% carbon dioxide.

Cells were detached using conventional cell culture techniques and 60000 suspended cells in 100 μL media were incubated with 2 μg of each of the following antibodies—Anti-Integrin alpha V [272-17E6] (Abcam, USA), Anti-Integrin alpha 5 [P1D6]—Azide free (Abcam, USA), Anti-Integrin beta 3 antibody [25E11], Anti-Integrin αV/β5 (P1F6) L (Santa Cruz Biotechnology, USA), Anti-Integrin αVβ6, clone 10D5 (Merck, USA)—for 30 minutes in a 37° C. incubator. Control antibody used was normal mouse IgG1 antibody (Santa Cruz Biotechnology, USA). The volume of the cell suspension was brought up to 250 μL for subsequent seeding in a 48-well tissue culture plate. Two days after antibody treatment, extracellular matrix was added into the media. Reprogramming of U251 cells followed a modified procedure, where a 3-antibody combination comprising Anti-Integrins αv, αvβ5 and αvβ6 was used. Two days after antibody treatment, the cells were transferred to ECM that had been pre-coated onto the culture wells. Extracellular matrices used are basement membrane matrix, Geltrex (Life Technologies, USA), laminin 332 and laminin 511 (Biolamina, Sweden). One day after addition of extracellular matrix, the media was changed to one of the following growth factors-supplemented media—1) SKP media made up of DMEM/F12 (Life Technologies, USA) supplemented with 1× B27 supplement (Life Technologies, USA), 40 ng/mL bFGF, 20 ng/mL EGF (Life Technologies, USA); 2) mTESR2 (Stemcell Technologies, USA).

Reprogramming to a Neuronal Phenotype

Cells were detached using conventional cell culture techniques and resuspended in Rin5f conditioned medium. ˜450,000 suspended cells in 300 μL media were incubated with 30 uL of each of the following antibodies—Anti-Integrin alpha V, Anti-Integrin alpha 5, Anti-Integrin beta 5 antibody, Anti-Integrin β6—for 5 minutes in a 37° C. incubator. The volume of the cell suspension was brought up to 3 mL with conditioned media, and cells were seeded by adding 500 μL of the suspension each, to six wells of a 24-well tissue culture plate coated with Geltrex (˜75,000 cells per well). After 24 h of culture, the cells were trypsinized and replated on TCP in SKP media or DMEM. After 72 h, cells of a neuronal morphology were observed growing among fibroblastic cells on the plate. The cells were fixed, stained with anti-βIII tubulin (Promega) and Hoeschst 33342, and viewed under the fluorescent microscope.

Specifically, cells were incubated with 1% BSA in PBST for 1 h with shaking to block unspecific binding. Cells were then incubated in primary antibody (anti-βIII tubulin, Promega) 1:1000 in 1% BSA in PBST, overnight. Subsequently, the cells were washed for 5 min in PBS. This was repeated twice. The cells were then incubated in 1 μg/mL secondary antibody for 1 h at room temperature and cells were washed thrice in PBS, in the dark. Finally cell nuclei were stained with Hoechst 33342 (1:5000).

RNA Isolation and cDNA Synthesis

Cells were lysed for RNA using TRIzol® Reagent (Invitrogen, USA) following manufacturer's instructions. Purified RNA was reverse transcribed into cDNA using iScript™ cDNA synthesis kit (Bio-Rad, USA). The reaction was carried out in a 20 μL volume containing 4 μL 5× iScript reaction mix, 1 μL iScript reverse transcriptase, RNA template (≦1000 ng total RNA) and nuclease free water (variable). The complete reaction mix was incubated for at 5 minutes at 25° C., 30 minutes at 42° C., 5 minutes at 85° C. followed by holding at 4° C. (optional).

Real-Time Reverse Transcriptase Polymerase Chain Reaction Analyses

Reaction was performed on iQ5 multi-colour real-time PCR detection system (Bio-Rad, USA) using the iTaq™ Universal SYBR® Green supermix (Bio-Rad, USA). The 20 μL volume reaction component includes 10 μL iTaq™ Universal SYBR® Green supermix, 1 μL of primer mix (5 μM forward primer, 5 μM reverse primer), 1 to 100 ng template and nuclease free water (variable).

The reaction conditions were as follows: 30 sec at 95° C., followed by 40 cycles of 15 sec at 95° C. and 30 sec at 60° C. Melting curve analysis was performed for 51 cycles, 6 sec each with a temperature increment of 0.5° C./cycle starting from 70° C. Relative quantization of target mRNA expression, normalized to an endogenous control and relative to a calibrator, was calculated using the mathematical expression for fold change 2-ΔΔCt (fold change).

Primers Design

PCR primers were designed for human gene expression detection and specifically exclude pseudogenes found in the human genome. Primers' sequences are listed in Table 1. The DNA oligonucleotides were synthesized by Integrated DNA Technologies (IDT) Singapore.

TABLE 1
Real-Time PCR Primer Sequences

| Target | Forward primer (5′-3′) | Reverse primer (5′-3′) |
|---|---|---|
| GAPDH | TTGACGCTGGGGCTGGCATT (SEQ ID | GTGCTCTTGCTGGGGCTGGT (SEQ ID |
| | NO. 1) | NO. 2) |
| ITGα2 | CCTACAATGTTGGTCTCCCAGA (SEQ ID | AGTAACCAGTTGCCTTTTGGATT (SEQ |
| | NO. 3) | ID NO. 4) |
| ITGα3 | TCAACCTGGATACCCGATTCC (SEQ ID | GCTCTGTCTGCCGATGGAG (SEQ ID |
| | NO. 5) | NO. 6) |
| ITGα5 | TCGTGTCCGCTAGTGCCTCC (SEQ ID | GATGCAGGCCACAGGGTTCC (SEQ ID |
| | NO. 7) | NO. 8) |
| ITGα6 | ATGCACGCGGATCGAGTTT (SEQ ID NO. | TTCCTGCTTCGTATTAACATGCT (SEQ |
| | 9) | ID NO. 10) |
| ITGαV | ATCTGTGAGGTCGAAACAGGA (SEQ ID | TGGAGCATACTCAACAGTCTTTG (SEQ |
| | NO. 11) | ID NO. 12) |
| ITGβ4 | AGCAGACCAAGTTCCGGCAGCA (SEQ ID | GCGCCATCAGCACTGTGTCCAC (SEQ |
| | NO. 13) | ID NO. 14) |
| ITGβ5 | TCTCGGTGTGATCTGAGGG (SEQ ID NO. | TGGCGAACCTGTAGCTGGA (SEQ ID |
| | 15) | NO. 16) |
| ITGβ6 | GCGAGAGAAGAAGCAGGCACAT (SEQ | AAAGAGCCGTTCCCGTGGTG (SEQ ID |
| | ID NO. 17) | NO. 18) |
| CDH1 | ACGCTGTGTCATCCAACGGG (SEQ ID | CCTCCTGGGTGAATTCGGGCTT (SEQ |
| | NO. 19) | ID NO. 20) |
| DAG1 | CTCTCTGTGGTTATGGCTCAGT (SEQ ID | CTGTTGGAATGGTCACTCGAAAT (SEQ |
| | NO. 21) | ID NO. 22) |
| EpCAM | GGACCTGACAGTAAATGGGGAACA | ACAACTGCTATCACCACAACCACA |
| | (SEQ ID NO. 23) | (SEQ ID NO. 24) |
| KRT18 | GGCATCCAGAACGAGAAGGAG (SEQ ID | ATTGTCCACAGTATTTGCGAAGA (SEQ |
| | NO. 25) | ID NO. 26) |
| LAMA3 | CACCTGCCAGCACTCAAGAG (SEQ ID | AGGGATCCTCAGTGTCGTCAA (SEQ |
| | NO. 27) | ID NO. 28) |
| LAMB3 | CAGCAGCTTGCGGAAGGT (SEQ ID NO. | TGTTTTATTCTCTCAAATCCCTCTTG |
| | 29) | (SEQ ID NO. 30) |
| LAMC2 | TCTCGGCTTCAGGGAGTCA (SEQ ID NO. | CGCTTTTTGTTTGATCCTCTTTG (SEQ |
| | 31) | ID NO. 32) |
| OCT4A | TGGAGAAGGAGAAGCTGGAGCAAAA | GGCAGATGGTCGTTTGGCTGAATA |
| Variant 1 | (SEQ ID NO. 33) | (SEQ ID NO. 34) |
| NANOG | CTGAGCTGGTTGCCTCATGT (SEQ ID | AAAGCAAGGCAAGCTTTGGG (SEQ ID |
| | NO. 35) | NO. 36) |
| SOX2 | GCGGGGGAATGGACCTTGTA (SEQ ID | TTCCTGCAAAGCTCCTACCGT (SEQ ID |
| | NO. 37) | NO. 38) |
| NESTIN | CTTCCCTCCGCATCCCGTCA (SEQ ID NO. | AAAGCCAGCATGTCACCCTCC (SEQ ID |
| | 39) | NO. 40) |
| CXCR4 | ACGGACAAGTACAGGCTGCAC (SEQ ID | CCAGAAGGGAAGCGTGATGACA (SEQ |
| | NO. 41) | ID NO. 42) |
| hTERT | AACCTTCCTCAGCTATGCCCG (SEQ ID | CAGCCGCAAGACCCCAAAGA (SEQ ID |
| | NO. 43) | NO. 44) |
| TP63 | CCAAAGCGAGGCACCCTTA (SEQ ID NO. | GGAGAGTAGGCTGCCATGAGG (SEQ |
| | 45) | ID NO. 46) |
| LIN28A | GAGCATGCAGAAGCGCAGATCAAA | TATGGCTGATGCTCTGGCAGAAGT |
| | (SEQ ID NO. 47) | (SEQ ID NO. 48) |
| CSPG4 | CTGTGGTGCTGACTGTCGTAGA (SEQ ID | GGTAGGGCAGGCCAAGGGTC (SEQ ID |
| | NO. 49) | NO. 50) |
| HIF1A | GAAAGCGCAAGTCCTCAAAG (SEQ ID | TGGGTAGGAGATGGAGATGC (SEQ ID |
| | NO. 51) | NO. 52) |
| PRRX1 | CTGATGCTTTTGTGCGAGAA (SEQ ID | ACTTGGCTCTTCGGTTCTGA (SEQ ID |
| | NO. 53) | NO. 54) |
| ZEB1 | GGGCGACCAAGAACAGGACT (SEQ ID | GTGTGGGACTGCCTGGTGAT (SEQ ID |
| | NO. 55) | NO. 56) |
| CDH2 | GGTGGAGGAGAAGAAGACCAGG (SEQ | GGCATCAGGCTCCACAGTGT (SEQ ID |
| | ID NO. 57) | NO. 58) |
| VIM | CCTTGAACGCAAAGTGGAATC (SEQ ID | GACATGCTGTTCCTGAATCTGAG (SEQ |
| | NO. 59) | ID NO. 60) |
| FN1 | TACTGGCCTGGAACCGGGAA (SEQ ID | ACCAGTTGGGGAAGCTCGTC (SEQ ID |
| | NO. 61) | NO. 62) |
| SNAIL | ACGGCCTAGCGAGTGGTTCT (SEQ ID | GATTGGGGTCGGAGGGCTTC (SEQ ID |
| (SNAI1) | NO. 63) | NO. 64) |
| Slug (SNAI2) | AGCTTTCAGACCCCCATGCC (SEQ ID | TGGCCAGCCCAGAAAAAGTTGA (SEQ |
| | NO. 65) | ID NO. 66) |
| TGFB1 | GGGCAGATCCTGTCCAAGC (SEQ ID NO. | GTGGGTTTCCACCATTAGCAC (SEQ ID |
| | 67) | NO. 68) |
| FGF2 | CGTGCTATGAAGGAAGATGGA (SEQ ID | TGCCCAGTTCGTTTCAGT (SEQ ID NO. |
| | NO. 69) | 70) |
| MMP9 | CGGAGCACGGAGACGGGTAT (SEQ ID | TTGGAACCACGACGCCCTTG (SEQ ID |
| | NO. 71) | NO. 72) |

GAPDH: Glyceraldehyde 3-phosphate dehydrogenase,
ITG: Integrin,
CDH1: E-cadherin,
DAG1: Dystrophin-associated glycoprotein 1,
EpCAM: Epithelial cell adhesion molecule,
KRT18: Keratin 18,
LAMA3: Laminin alpha 3,
LAMB3: Laminin beta 3,
LAMC2: Laminin gamma 2,
OCT4A: Octamer-binding transcription factor 4A,
NANOG: homeobox transcription factor Nanog,
SOX2: SRY (sex determining region Y)-box 2,
NES: Nestin,
CXCR4: Chemokine (C—X—C motif) receptor 4,
hTERT: human Telomerase reverse transcriptase,
CSPG4: Chondroitin sulfate proteoglycan 4,
HIF1A: Hypoxia inducible factor 1 alpha subunit,
PRRX1: Paired related homeobox 1,
ZEB1: Zinc finger E-box binding homeobox 1,
CDH2: N-cadherin,
VIM: Vimentin,
FN1: Fibronectin 1,
TGF-B1: Transforming growth factor beta 1,
FGF2: fibroblast growth factor 2 (basic),
MMP9: Matrix metallopeptidase 9.

Forced Expression of Integrins by Lentiviral Methods

DNA encoding human integrins were amplified from human genomic DNA and cloned into a third-generation lentiviral expression plasmids (Life Technologies). Lentivirus was packaged by co-transfection of lentiviral expression plasmids with the 3rd generation packaging plasmids pLP1, pLP2, and pLP/VSVG (Life Technologies) with Lipofectamine 2000 (Life Technologies) into 14 cm plates of 293FT cells. Medium was changed after 24 hours, and supernatants were harvested 72 hours after initial transfection, briefly centrifuged at 1000 g for 5 mins to remove cellular debris, and ultra-centrifuged at 32,000 rpm 4° C. for 1 hour to concentrate the virus into a pellet. Pellets were re-suspended in Optimem (Gibco) and titers were determined with an infectivity assay using HT1080 cells.

IMR90 lung fibroblasts were infected with lentivirus encoding the respective integrins at a multiplicity of infection of 5 and stably selected with a drug (Blasticidin, Zeocin). Over-expression of integrins in IMR90 lung fibroblast post-infection was confirmed with quantitative PCR.

Nanotopographical Scaffold

The nanotopographical scaffold comprised of uniform arrays of silica nanoparticles of two sizes, (66±6) nm and (414±37) nm, obtained by assembly of the particles onto glass coverslips followed by sintering. Silica nanoparticles were prepared in a typical base-catalyzed hydrolysis of tetraethylorthosilicate (TEOS) using ethanol as a medium.

The final concentration of ammonia was varied in order to obtain different final sizes of the silica nanoparticles. For synthesis of 414 nm particles, 1 ml of TEOS (98%, Aldrich) was first added to 4 ml of absolute ethanol (Fisher Chemical) and mixed to a homogenous solution with a stirrer bar. This solution was then added dropwise to a mixture of 9 ml of 25% ammonia solution (Merck) and 46 ml of absolute ethanol. The reaction broth was stirred overnight for complete base-catalyzed hydrolysis of TEOS.

For synthesis of 66 nm particles, 1 ml of TEOS was first added to 4 ml of absolute ethanol and mixed homogenously with a stirrer bar. This solution was then added dropwise into a mixture of 3 ml of 25% ammonia solution and 52 ml of absolute ethanol. The reaction broth was stirred overnight for complete base-catalyzed hydrolysis of TEOS.

The nanotopographical substrate was prepared via thermal evaporation of the nanoparticle suspension obtained above on a glass coverslip, causing its self-assembly into a closely packed array. The substrates were sintered to ensure complete adhesion of particles to the glass coverslip.

Western Blotting

Isotype-treated and antibodies-treated cells were detached using routine cell culture techniques and cell pellets obtained were washed with PBS. The washed cell pellet was re-suspended in an 80 μL ice-cold RIPA lysis buffer (Santa Cruz, USA), mixed, The cell lysate was centrifuged at 4° C. for 10-15 min at 15000×g to pellet cellular debris. Cellular lysate (supernatant) was then collected and placed on ice. Protein concentration was determined using Bio-Rad Dc protein assay (Bio-Rad, USA) according to the manufacturer's instructions.

Western blotting of the cell lysates was carried out according to the standard protocols. An equal amount of protein (20 μg) was loaded into the NuPAGE® 4-12% Bis-Tris gel (Invitrogen, USA) and ran at 200 Volt for 35 min. The gel was then blotted onto a nitrocellulose membrane which was subsequently probed for the protein of interest with a modified antibody.

The membrane was blocked with 5% non-fat dry milk (Bio-Rad, USA) in 0.1% Tween20 (Promega, USA) Tris-buffered saline (1^{st} Base, Singapore) (TBST) for 20 min at room temperature with shaking. Prior to washing the membrane thrice, 5 min each time, with TBST buffer, it was incubated with primary antibody (1:200 dilution) in 5% non-fat dry milk TBST overnight at 4° C. under gentle shaking. Next, the membrane was incubated with horseradish peroxidase-conjugated secondary antibody (Santa Cruz, USA), at 1:5000 dilution, for 2-3 hr at room temperature. Blot was washed with TBST buffer for 3 times, 5 min each. Membrane was subsequently incubated with 500 μL of ECL™ Prime reagent (GE Healthcare, UK) for 5 min before imaging.

### EXAMPLE 1

Reprogramming to Stem-Cell Phenotypes by Integrin Inhibition

Fibroblasts

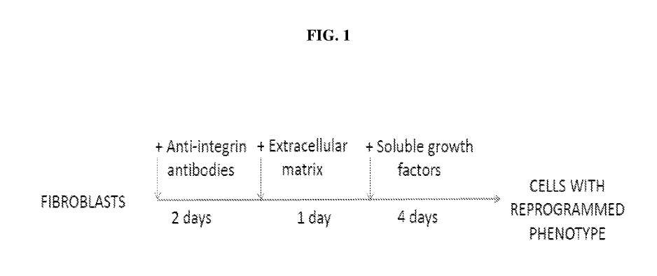

The timeline for the method of reprogramming fibroblasts to stem cell-like phenotypes by selective integrin inhibition and ligation (SIIL) is shown in FIG. 1.

In designing a method to reprogram fibroblasts by controlling their interactions with the ECM, the transitions that occur between epithelial and mesenchymal cell phenotypes (EMT/MET) and the possible role of the ECM in controlling these interactions was taken into account. Epithelial cells form the outer covering of organs, lining of glands and ducts, and due to their barrier role, exhibit tight junctions and apical-basolateral polarity. Mesenchymal cells on the other hand, are the main cell type of connective tissue, exhibit a bipolar morphology, and are more motile. It was hypothesized that, in the course of transforming a mesenchymal phenotype to an epithelial phenotype (MET), an intermediate metastable phenotype with higher differentiation potential would result. Thus, the first step in the strategy for reprogramming fibroblasts, a mesenchymal cell type, was to effect an MET.

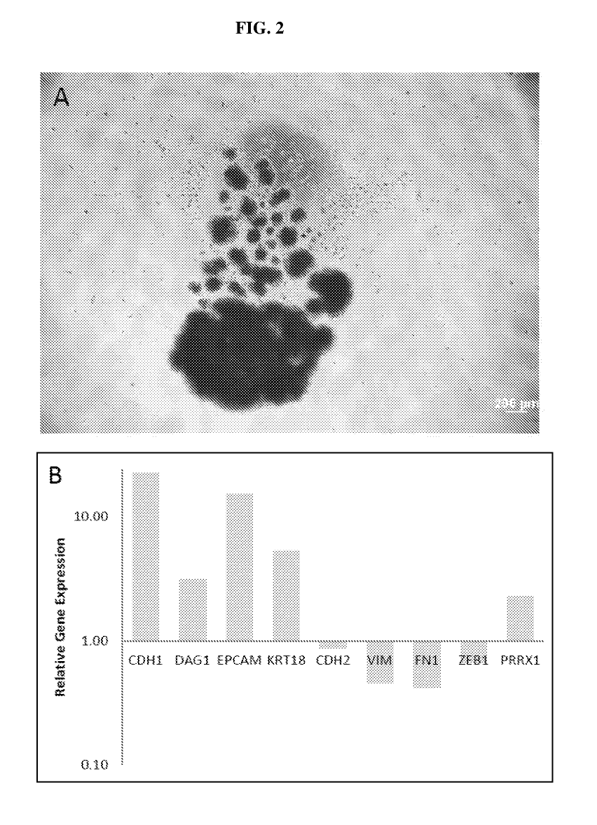

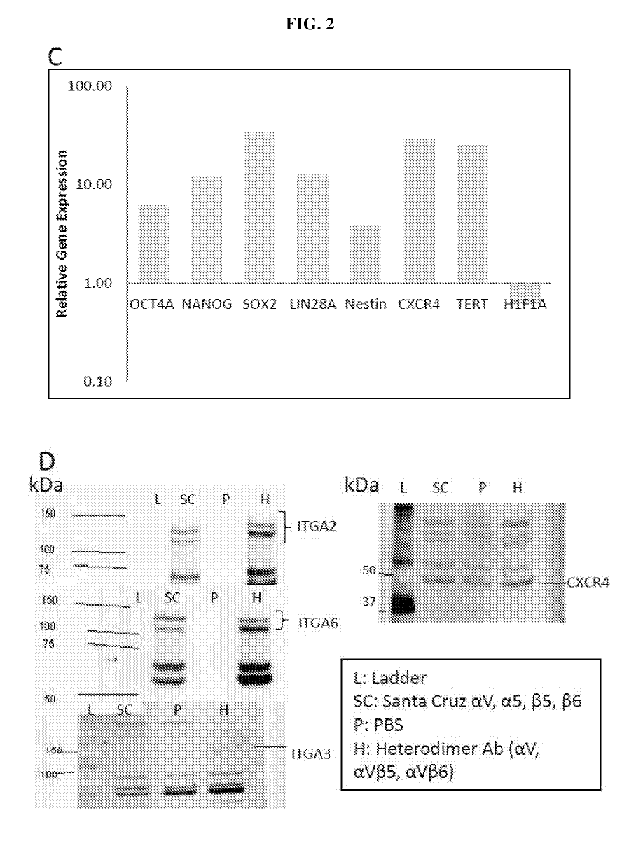

In this study, a MET in fibroblasts was effected by applying antibodies against integrins that involved in the reverse process of EMT. These are generally integrins that bind to arginine-glycine-aspartic acid (RGD) ligands, present in ECM such as fibronectin and vitronectin. Permutations of these antibodies led to gene expression changes that were suggestive of MET. When the optimal combination of antibodies (anti-integrins alpha v, alpha 5, beta 5, beta 6 and beta 3) was applied, the cells largely detached from the matrix to form spherical cell clusters (SCC) within 2 days of culture and exhibited gene expression changes that were suggestive of MET (FIGS. 2A and B). Concomitantly, a panel of pluripotent and stem cell associated markers were also upregulated (FIG. 2C). This supported the notion that inhibition of the selected set of integrins in fetal lung fibroblasts had led to an intermediate phenotype with MET and stemness characteristics.

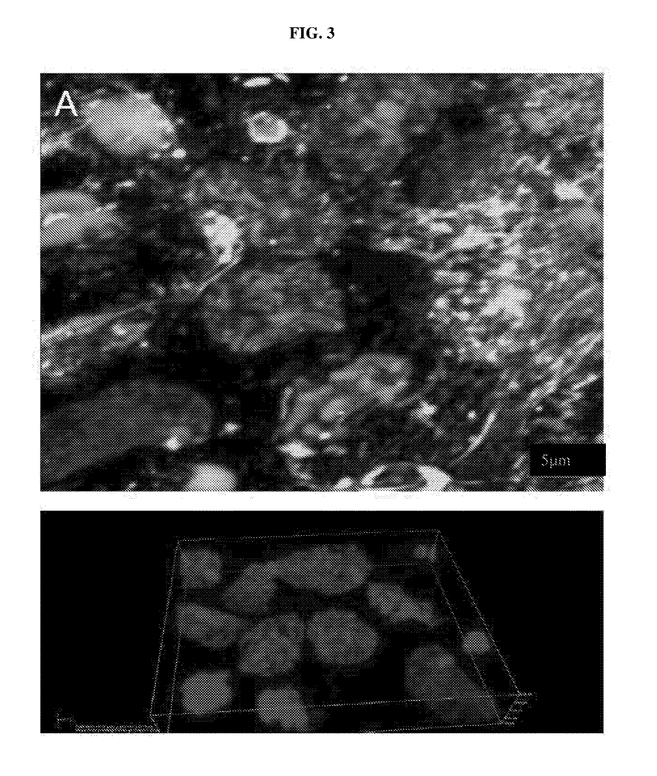

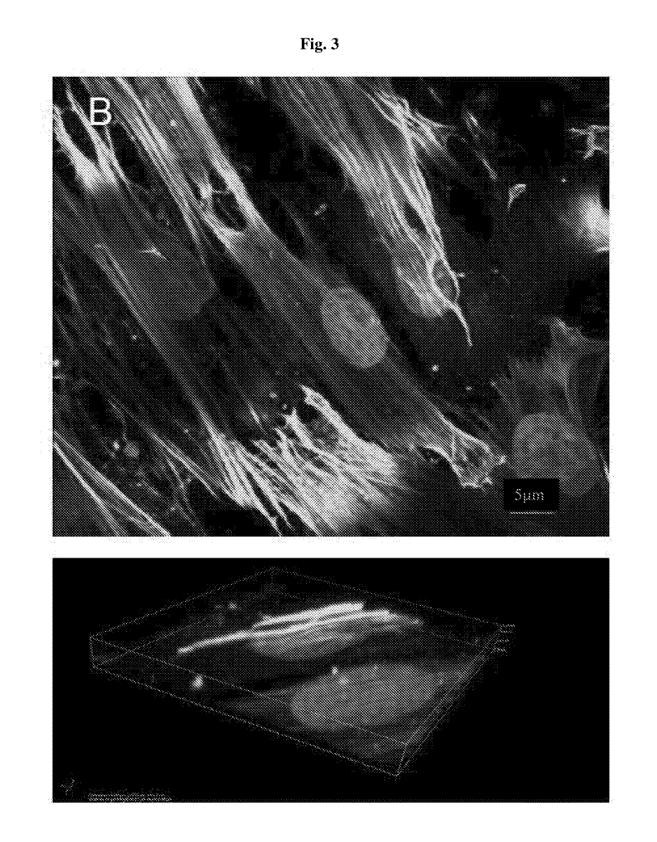

Interestingly, changes in the expression of integrins were also observed at the protein level (FIG. 2D). While the original fibroblasts primarily expressed integrins that act as receptors to RGD ligands (alpha V integrins), other integrins acting as receptors to collagen and laminin ligands (alpha 6, beta 4 and alpha 2) were expressed in the intermediate phenotype. The latter also showed higher expression of integrin alpha 3 and CXCR4, though to a lesser extent. The high-resolution confocal microscope images (FIG. 3) show the differences in chromatin structure upon induction of the fetal lung fibroblasts to the intermediate phenotype. Induced IMR90 fibroblasts (the intermediate phenotype) exhibit larger condensed heterochromatin domains, whereas the control fibroblasts appear to contain finer heterochromatin. The induced fibroblasts also exhibit a more irregular nuclear shape with a lower aspect ratio, as compared to the regular, oval shape of the control fibroblasts.

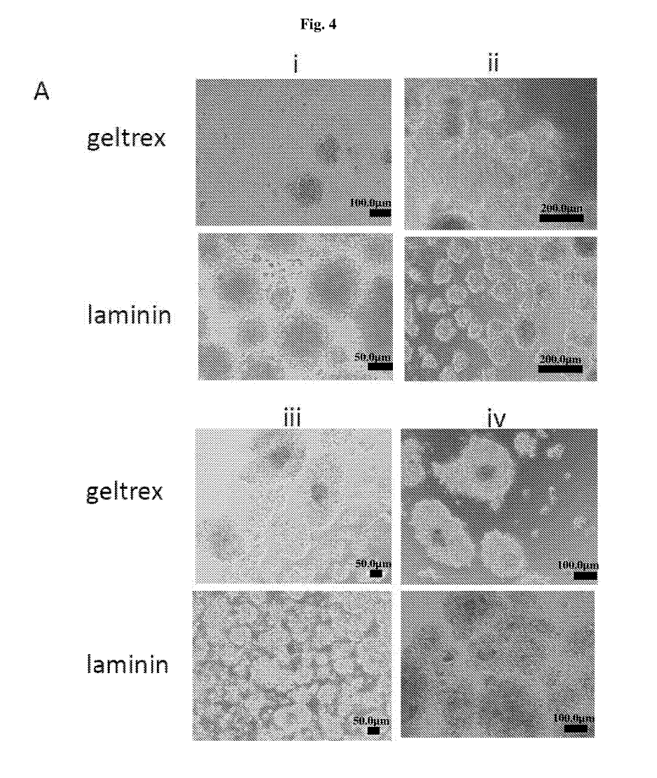

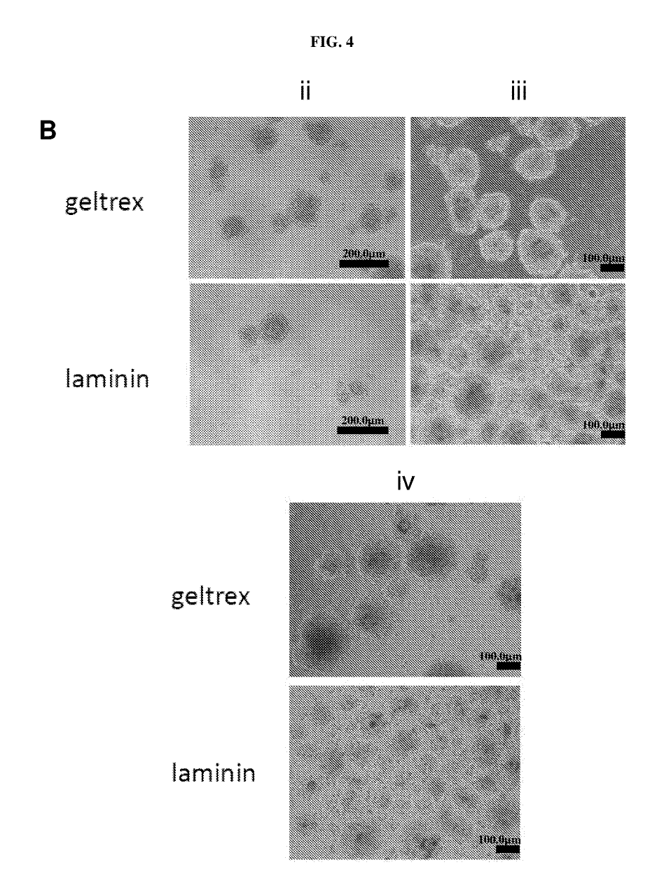

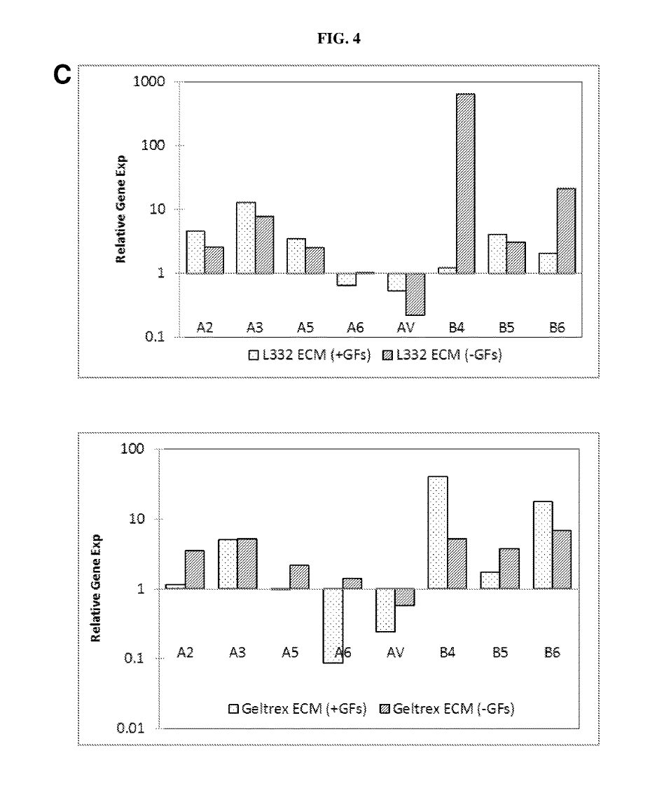

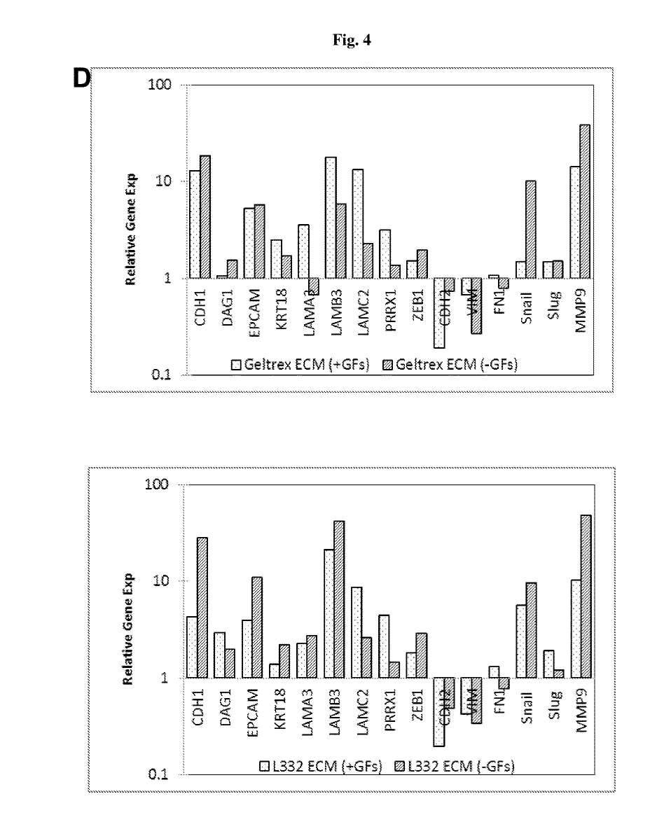

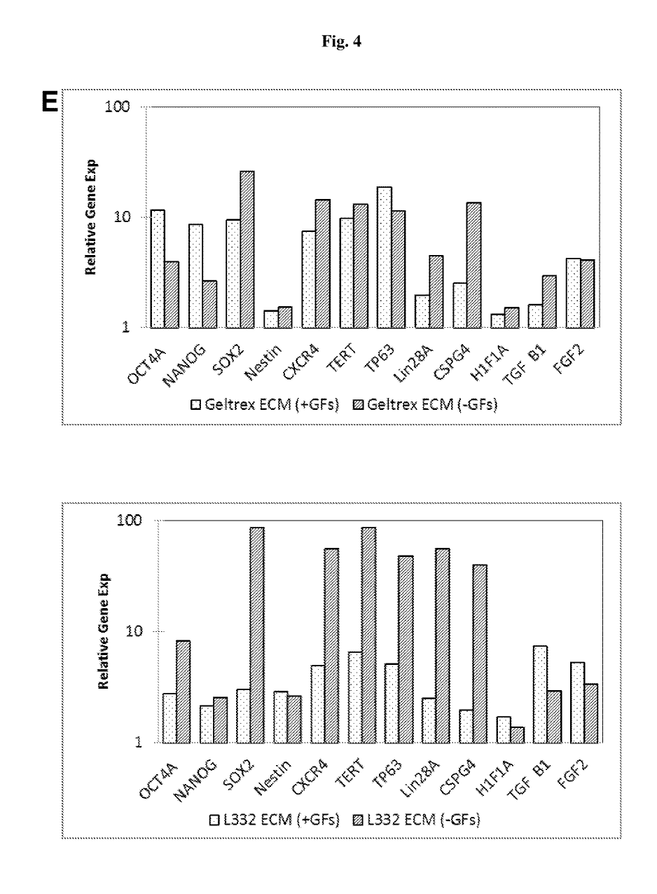

After 2 days of culture, two ECM types—a basement membrane matrix, Geltrex and Laminin 332 in solution form, were added to the SCC cultures. This step was aimed at providing a selected set of ligands to ligate the integrins expressed by the induced intermediate phenotype (including the newly expressed alpha 2 and alpha 6 integrins). After overnight culture, most of the clusters had attached to the plate (FIG. 4Ai). At this point, the media was changed to fresh medium with ECM but not containing antibodies. B27 medium and growth factors (FGF2, EGF) was used for one set of cultures, while fresh DMEM medium was used for the second set.

For the cells cultured in media with growth factors, the compact, cell-dense SCC morphology gradually changed to clusters of cells with a progressively growing translucent outer ring and receding cell-dense core (FIG. 4Aii). By day 4 of the media change, the cell-dense core had almost disappeared for most of the clusters. Some of the cells were harvested for gene expression analysis (FIG. 4C-E), while the rest were used for the cell expansion and differentiation experiments.

The first step of the reprogramming approach relied on the ability to induce an MET in fibroblasts by selective integrin inhibition, leading to an intermediate phenotype (that took the form of spherical cell clusters) and expression of additional integrins capable of ligating basement membrane (laminin, collagen) ligands, in addition to the RGD binding integrins already expressed in fibroblasts. Upon induction, the cells exhibited reduced cell-ECM interactions, enhanced cell-cell interactions and detached from the substrate. The intermediate phenotype, which expresses a wider range of integrins than the original fibroblasts, is suggested to be a phenotype that is ‘primed’ towards reprogramming.

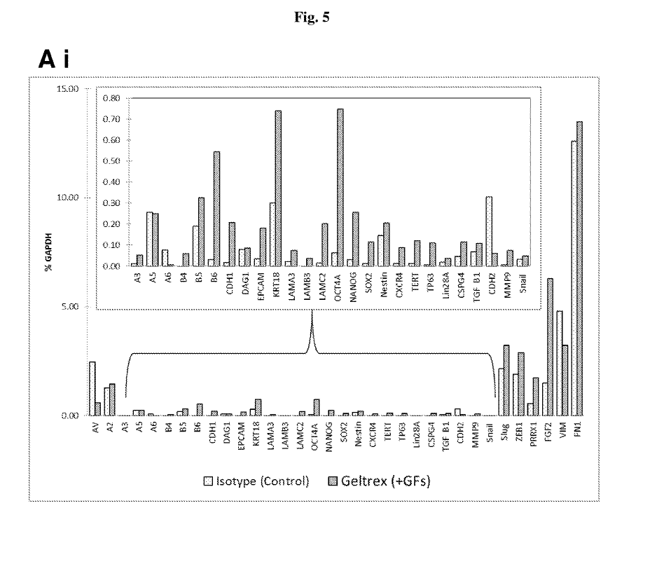

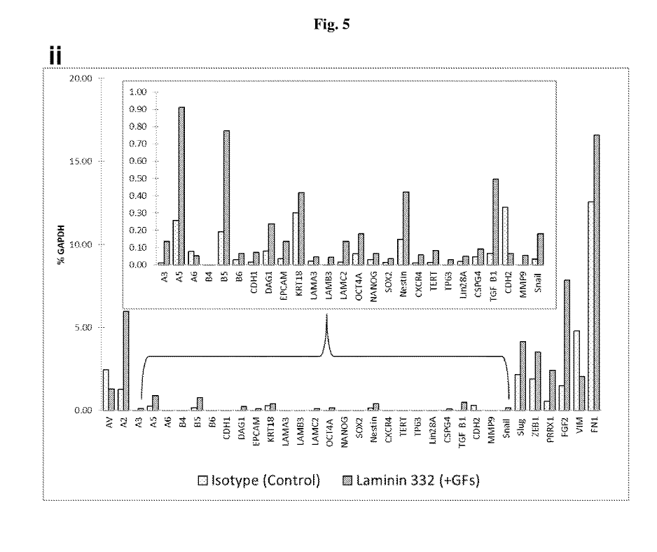

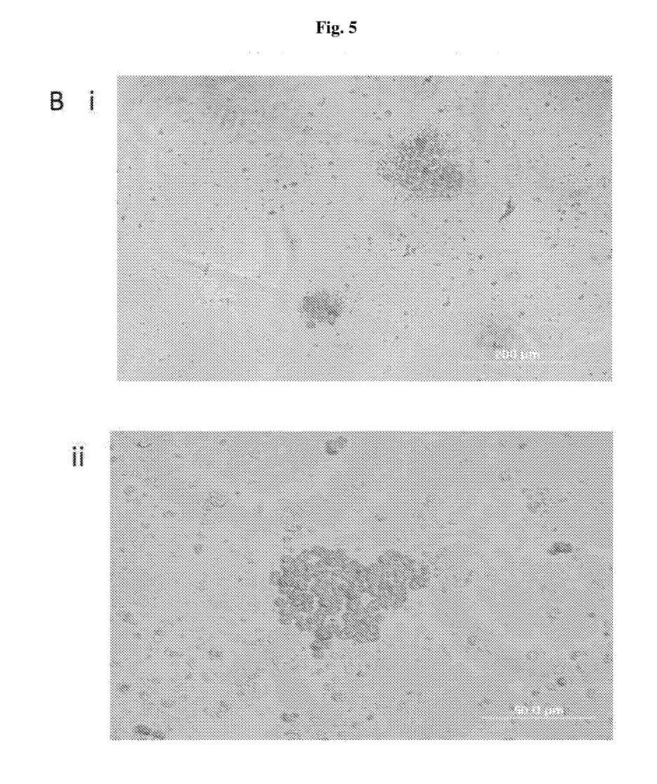

The second step of the reprogramming approach provides the second level of integrin selection, i.e. selective integrin ligation. Different ECM types provide different types of ligands that bind to selected integrins. For example, Laminin 332 is reported to bind to integrins alpha 2 and alpha 6. The commercial basement membrane matrix, Geltrex, in addition to containing laminins that bind the integrins alpha 2 and alpha 6, also contains other ECM components such as collagen and entactin that bind to other integrins. Experimenting with the Laminin 332 and Geltrex, it was found that the resulting induced stem cell phenotype depended on the specific ECM used (FIG. 5).

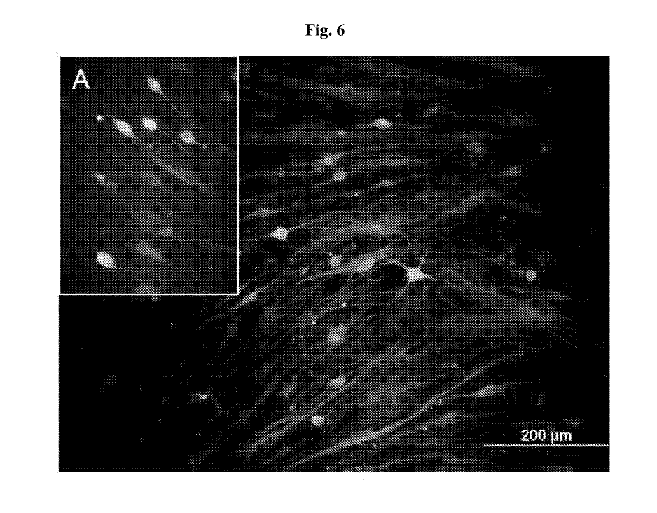

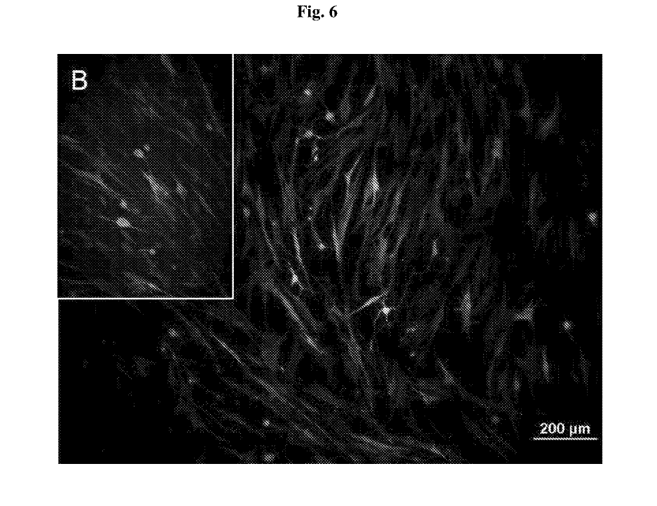

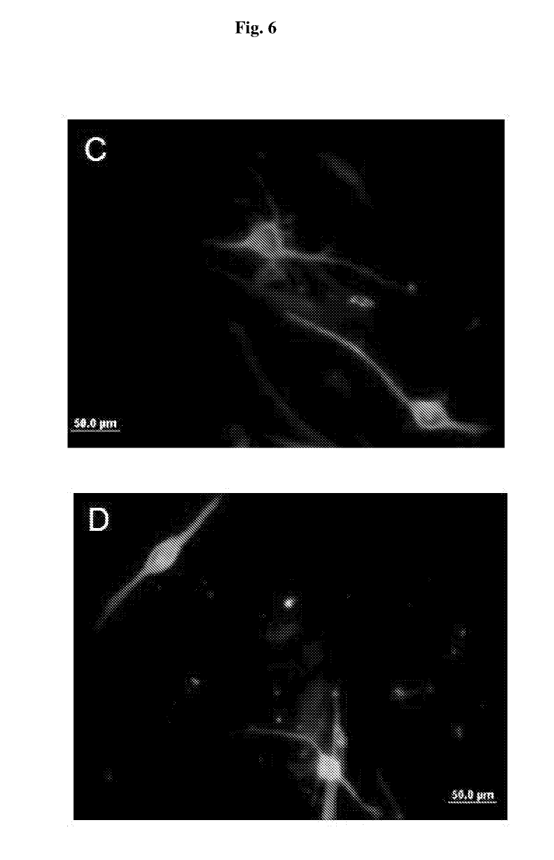

In addition to reprogramming fibroblasts to stem cell-like phenotypes, the method of the present invention could be used to reprogram fibroblasts directly to other somatic cell types, such as neurons. A similar combination of anti-integrin antibodies was used to induce the fibroblasts to the intermediate phenotype, in the presence of conditioned medium from a rat insulinoma (Rin5f) cell line. (Pancreatic beta cells secrete growth factors such as nerve growth factor) Antibody-treated fibroblasts were cultured on plates coated with Geltrex. After 24 hours of culture, the cells were detached from the plates using trypsin, and reseeded on tissue culture plate in either SKP media or DMEM. In all cases, after about 72 h of culture, a large number of cells had adopted a neuronal morphology that stained positive for the neuronal marker, beta-III tubulin. (FIG. 6).

Glioma Cell Line

In the context of cancer, reprogramming of tumor cells to less differentiated, more stem-like phenotypes afford them a mechanism to gain stem cell characteristics such as self-renewal and ability to differentiate to parenchymal cells, characteristics which may support cancer metastasis.

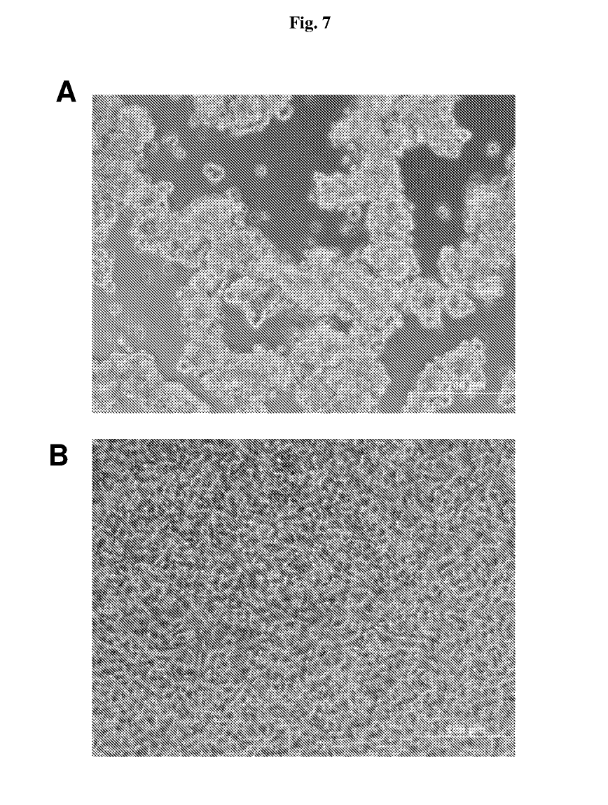

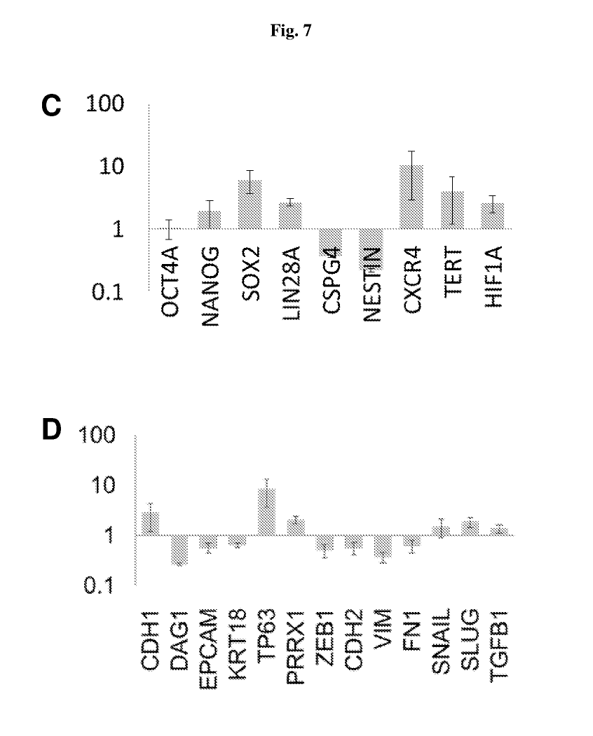

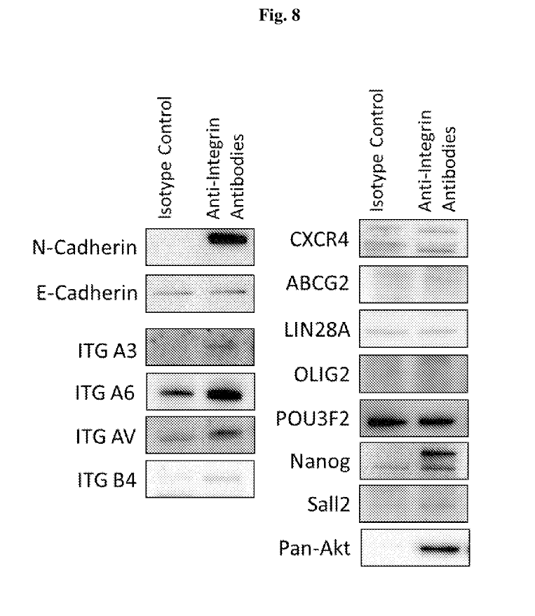

A 3-antibody combination comprising the integrins αv, αvβ5 and αvβ6 was applied to a glioma cell line, U251 for 2 days. FIG. 7 shows the respective morphologies of the antibody treated cells (A) and isotype controls (B), as well as changes in the expression of various stemness (C) and MET (D) markers, normalized against the corresponding isotype controls. Immunoblotting showed an upregulation of stemness markers at the protein level (FIG. 8). In addition, the repertoire of integrin expression appeared to be increased by antibody treatment (FIG. 8).

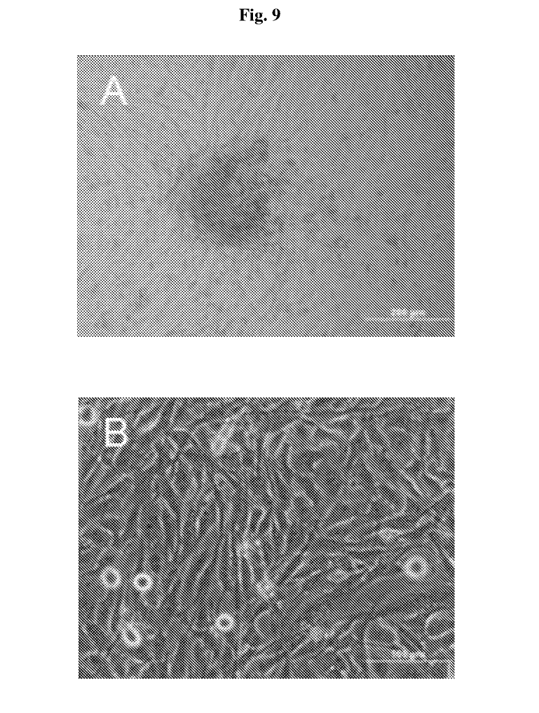

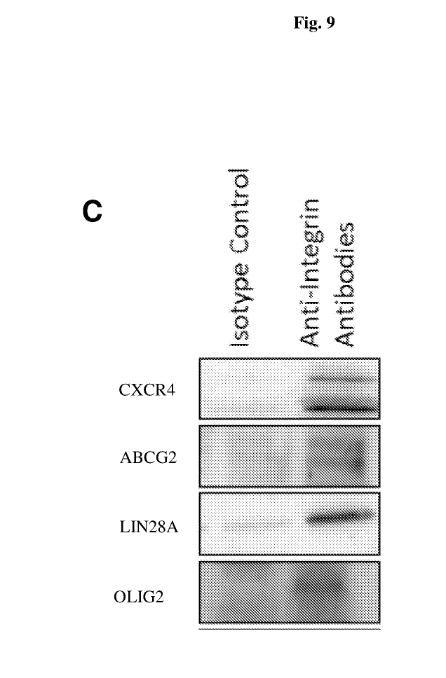

After 2 days, the cells were transferred to culture wells that had been pre-coated with Laminin 511. The replated cells attached and spread on Laminin 511 as depicted in FIG. 9 for the AB treated cells (A) and isotype control (B). The antibody treated and replated cells exhibited upregulation of several stemness markers, such as CXCR4, ABCG2, Lin28 and OLIG2 in comparison to the corresponding isotype control (C).

### EXAMPLE 2

Reprogramming by Integrin Overexpression

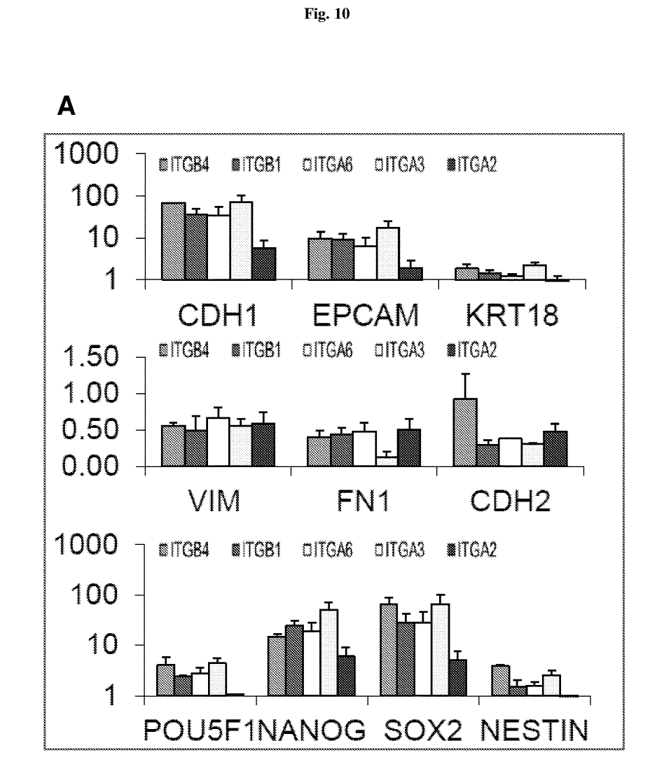

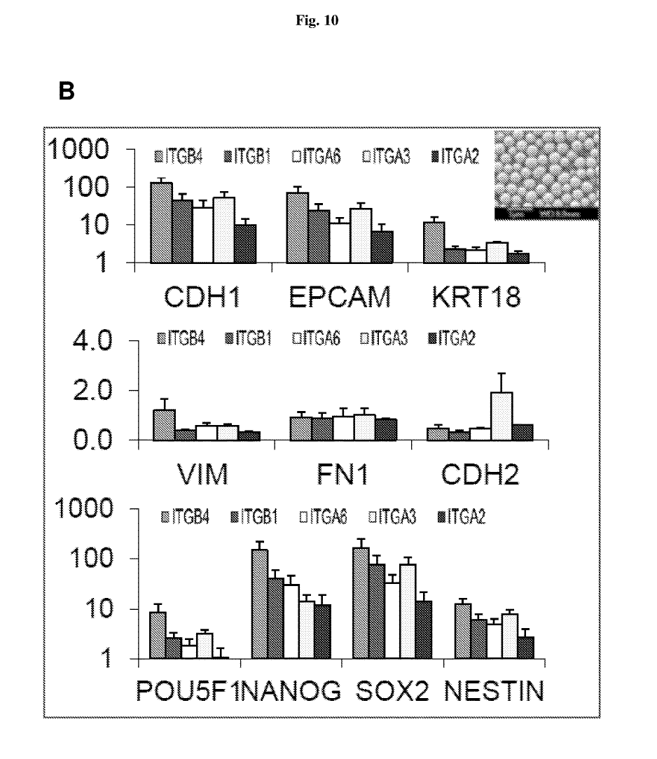

As an alternative method to reprogramming cells by integrin inhibition, appropriate integrins may also be overexpressed to obtain the intermediate phenotype, optionally in conjunction with a nanotopographical scaffold. Overexpression of the integrins α2, α3, α6, β1 and β4 led to increased expression of both epithelial and stemness markers, and decrease in expression of mesenchymal markers. The effect was more pronounced when the cells were cultured on a geltrex-coated nanotopographical substrate as compared to geltrex-coated coverslip (FIG. 10).

### EXAMPLE 3

Reprogramming to Other Cell Types by Integrin Inhibition

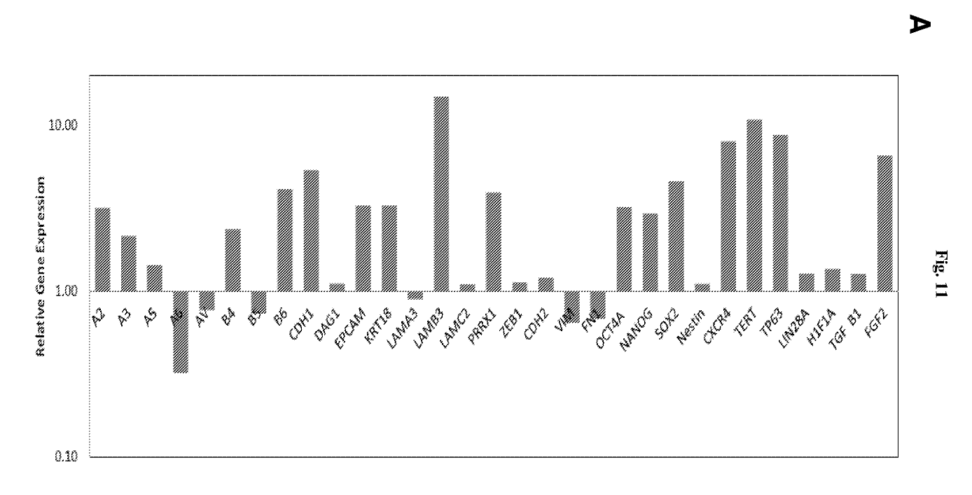

Inhibition of integrins using antibodies can also be used to induce the intermediate phenotype in other cell types. This was demonstrated for the case of human dermal fibroblasts and dermal papilla cells, where application of the antibodies similarly led to an induction of MET (increased expression of epithelial and decreased expression of mesenchymal markers) and upregulation of stem cell markers. (FIG. 11).

### EXAMPLE 4

Addition of Other Extracellular Matrices and Growth Factors During Integrin Ligation

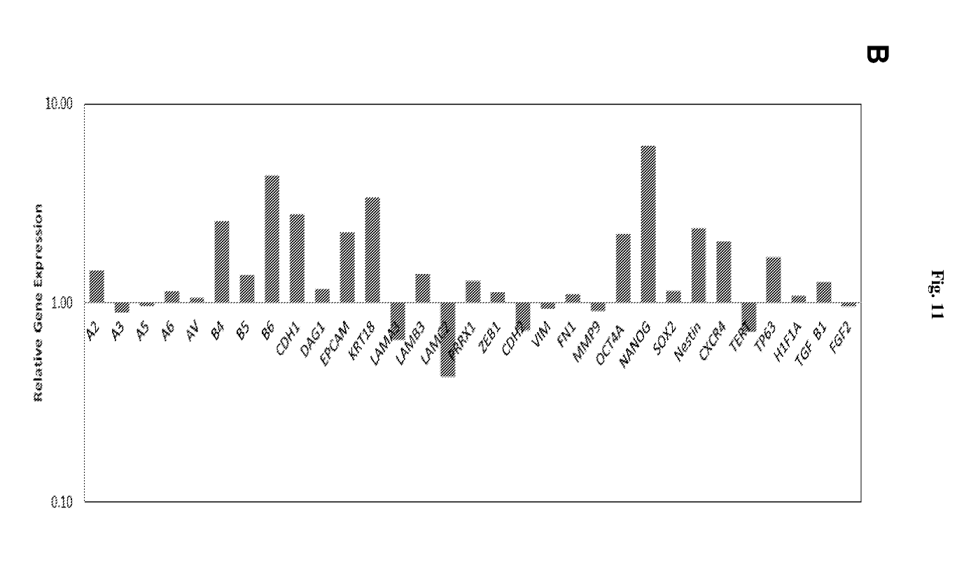

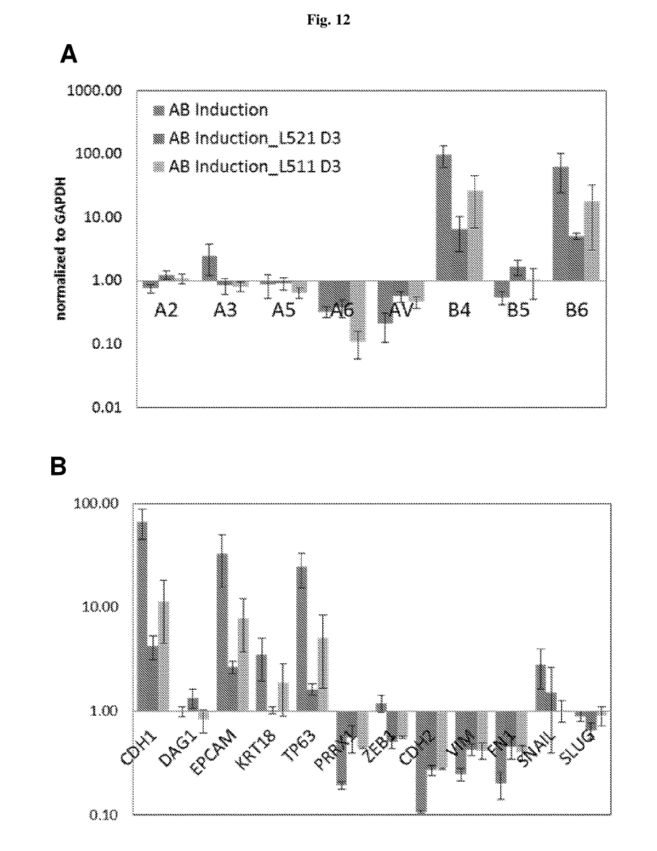

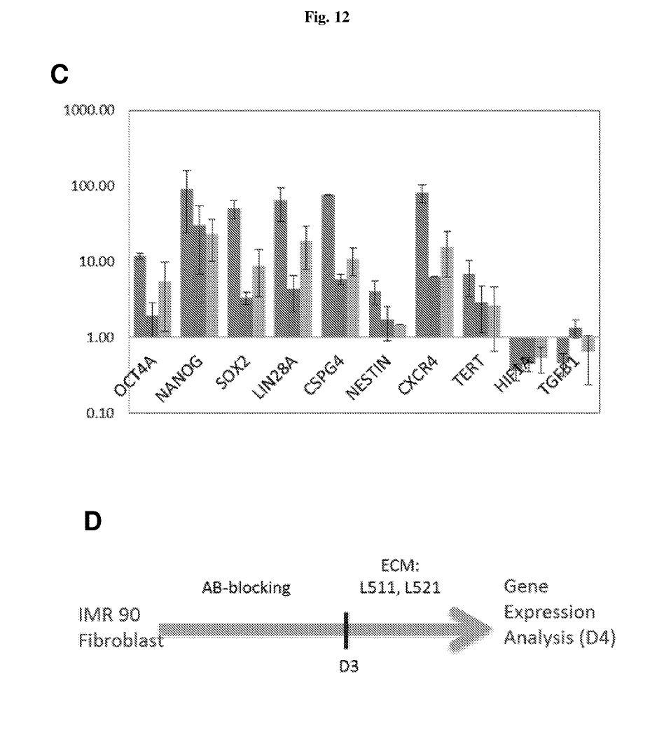

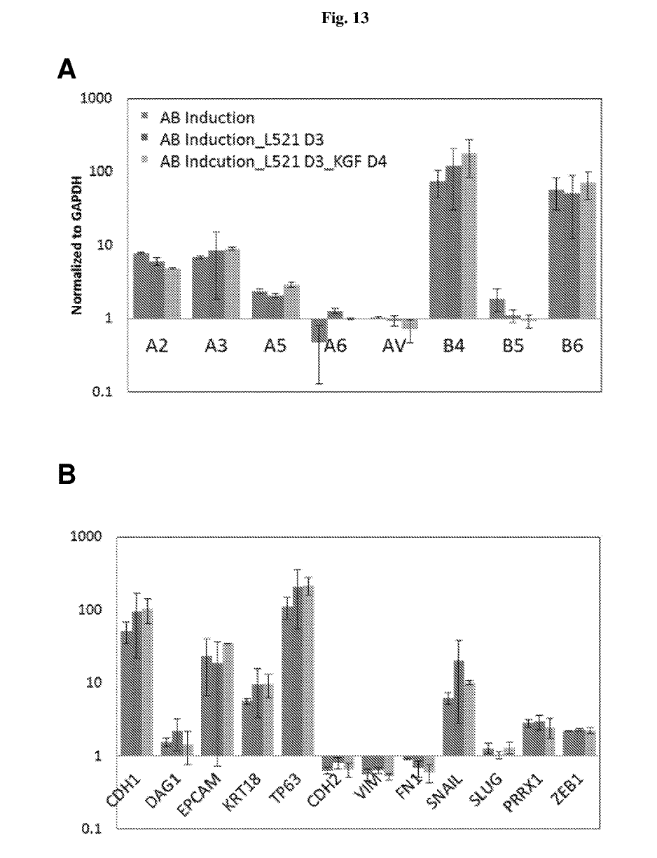

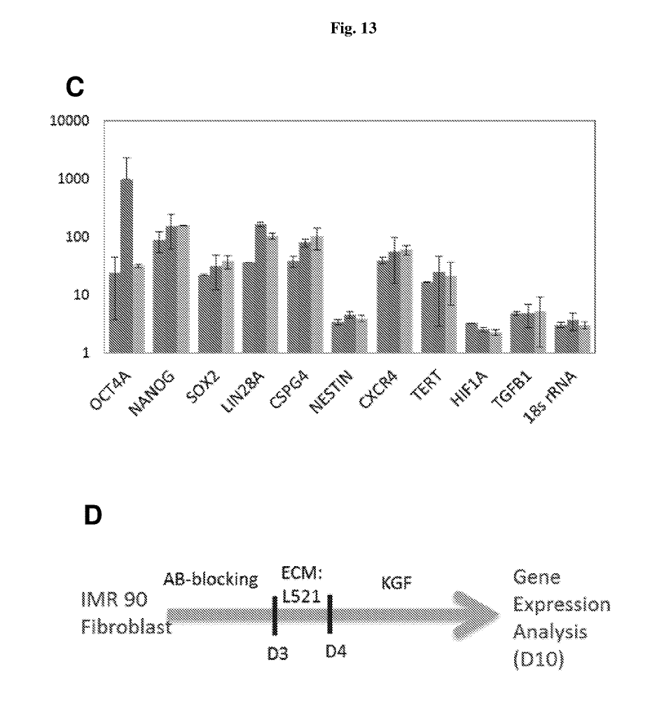

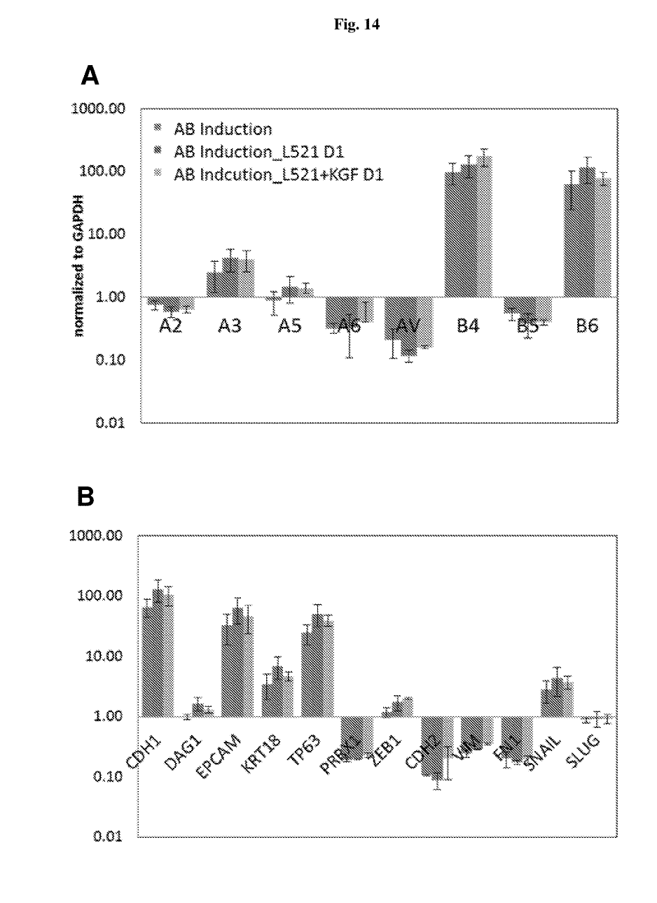

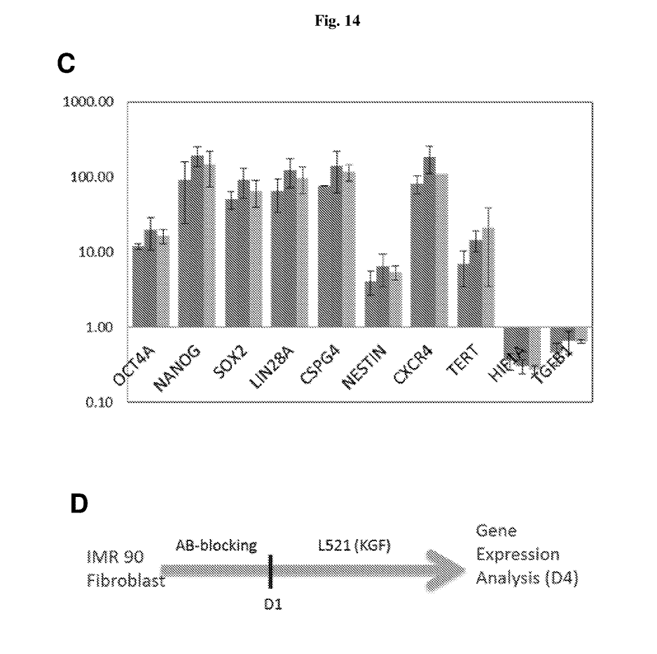

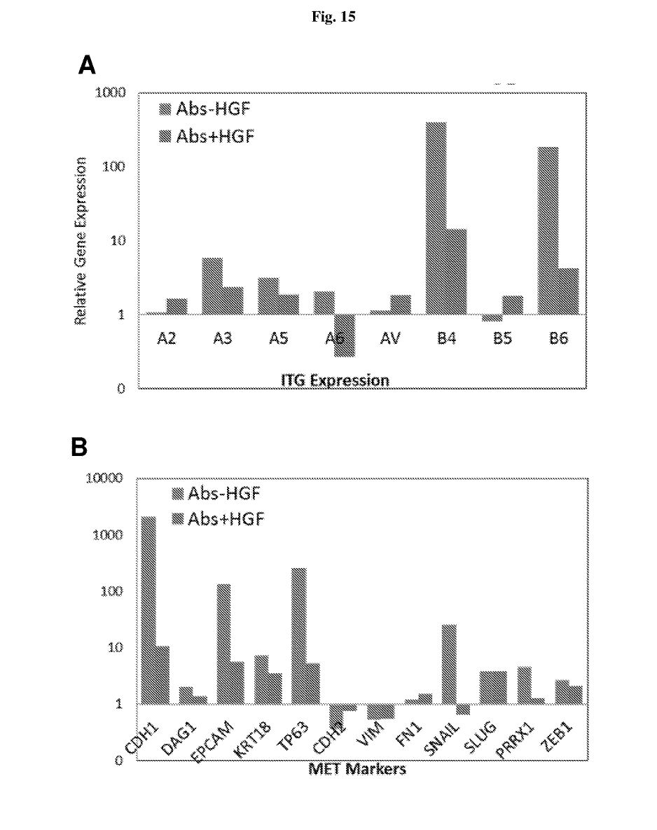

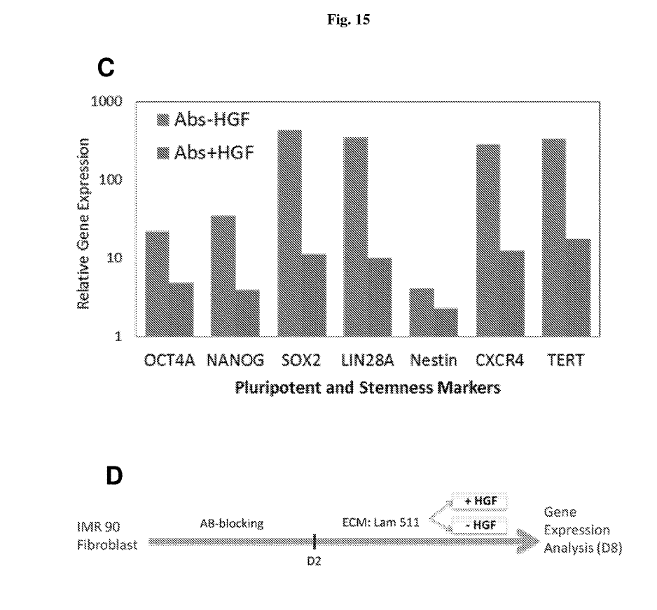

Additional experiments were performed to investigate the use of other extracellular matrices (Laminins 511 and 521) and growth factors (keratinocyte growth factor, KGF and hepatocyte growth factor, HGF) during the integrin ligation step. The results suggest that integrin ligation by presentation of both laminins positively influenced the expression of MET and stemness markers, provided the laminins were presented for a long enough period of time (FIG. 12-15). Introduction of KGF did not appear to significantly affect the level of the markers that were analysed. However, introduction of HGF reduced expression of both MET and stemness markers, which is postulated to be a result of EMT (FIG. 15).

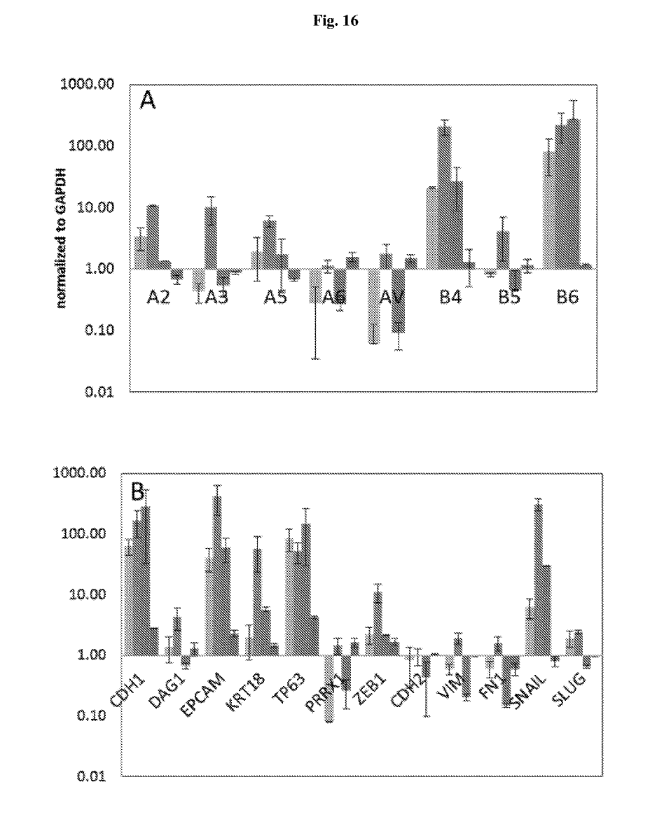

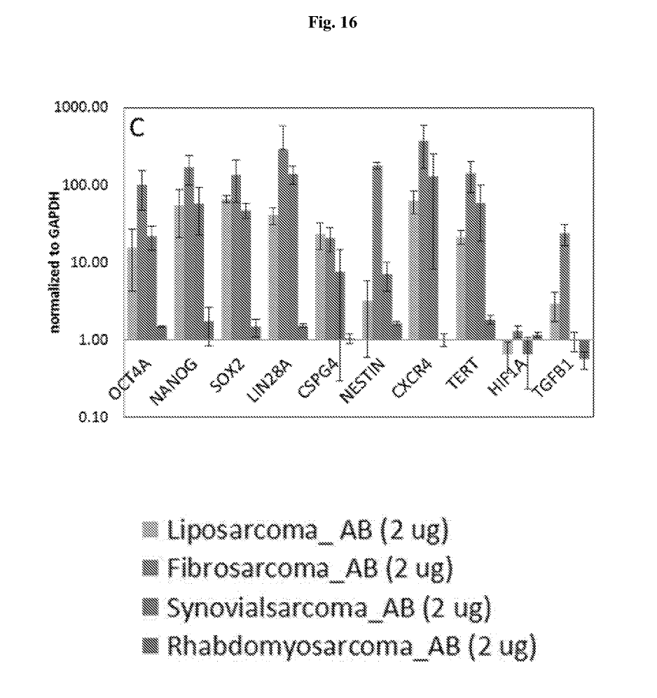

When the process of induction (integrin inhibition by antibody blocking) was applied to a panel of soft tissue sarcomas (ATCC® TCP-1019™), the results were comparable to those obtained for the fetal lung fibroblast, IMR90 cell line, i.e. increased expression of MET and stemness markers and upregulation of gene expression for the integrins beta 4 and beta 6 (FIG. 16). The induction of stemness suggests that the methodology of the present invention may be useful to derive cancer stem cells (CSCs) from cancer cell lines or primary cancer cells. Such induced CSCs would be valuable for cancer drug screening/assays.

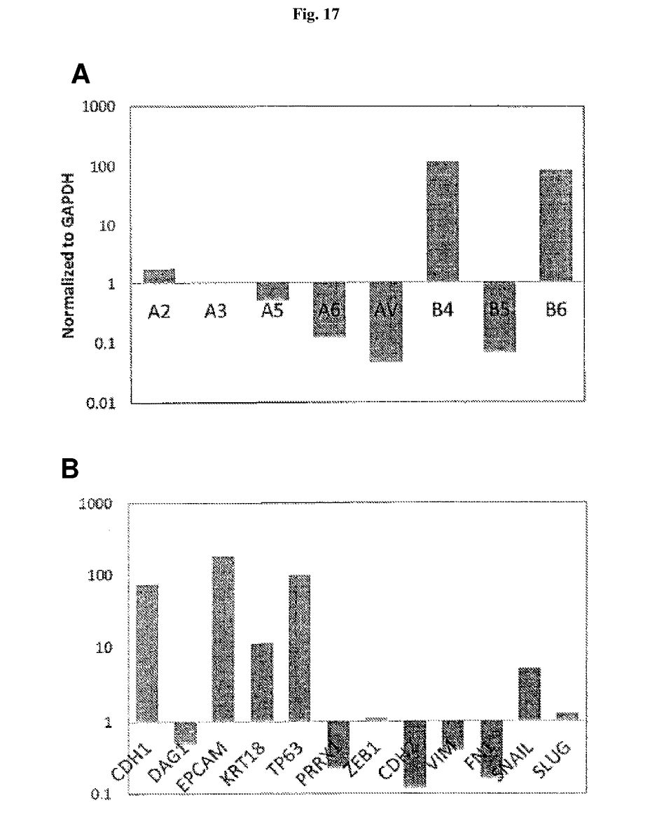

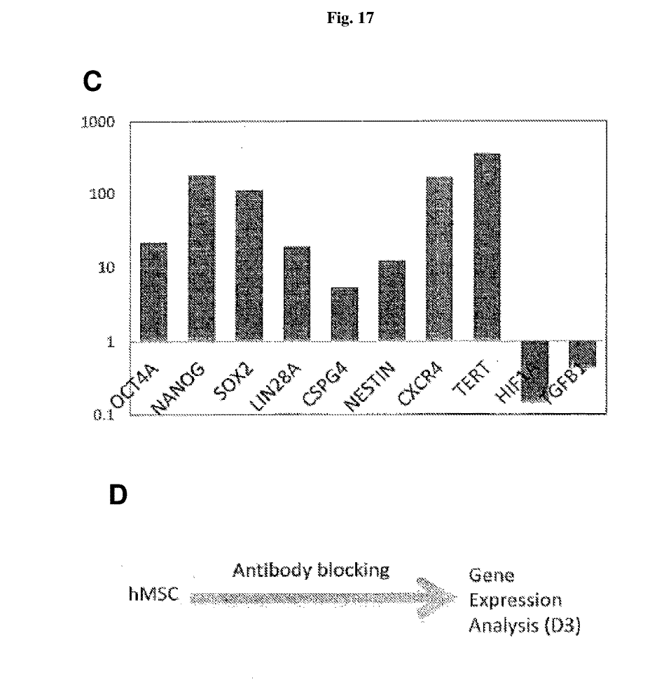

When the same process of integrin inhibition by antibody blocking was applied to human mesenchymal stem cells, hMSC (bone marrow, DV Biologics), the results were comparable to those obtained for the IMR90 cells, i.e. increased expression of MET and stemness markers and upregulation of gene expression for the integrins beta 4 and beta 6 (FIG. 17).

1. A method for reprogramming a first cell type to an intermediate cell of a second cell type, comprising the step of:
contacting said cell with a first agent to modulate an integrin profile in the first cell type to provide said intermediate cell of said second cell type.

2. The method of claim 1, wherein the first agent is at least one integrin ligand to provide said intermediate cell of said second cell type, optionally wherein the at least one integrin ligand is an extracellular matrix (ECM) protein or anti-integrin antibody.

3. (canceled)

4. The method according to claim 2, wherein the ligand is an anti-integrin antibody or fragment thereof.

5. The method of claim 4, wherein the anti-integrin antibody or fragment thereof is an integrin inhibitor, optionally wherein said integrin inhibitor inhibits one or more integrin subunits selected from the group consisting of α1, α2, α3, α4, α5, α6, α7, α8, α9, α10, α11, αv, αD, αL, αM, αX, αE, αIIb, β1, β2, β3, β4, β5, β6, β7 and β8.

6. (canceled)

7. The method according to claim 5, wherein said integrin inhibitor binds to an integrin subunit selected from the group consisting of αv, α5, β1, β3, β5, β6, and combinations thereof, optionally wherein the combination of subunits is αvβ5 or αvβ6.

8. (canceled)

9. The method according to claim 1, wherein said first cell type is contacted with said one or more integrin ligands for a period sufficient to alter gene expression, optionally wherein the period is selected from the group consisting of at least about 1 minute, about 3 minutes, about 5 minutes, about 10 minutes, about 15 minutes, about 30 minutes, about 60 minutes, about 90 minutes, about 2 hours, about 24 hours, about 2 days or about 5 days.

10. (canceled)

11. The method of claim 1, wherein the first agent is a nucleic acid molecule, wherein contacting said cell with said nucleic acid molecule alters the expression of at least one integrin gene, thereby modulating the integrin profile in said cell.

12. The method of claim 11, wherein the nucleic acid molecule is an expression vector that comprises at least one integrin gene operably linked to a promoter to overexpress said integrin gene in said cell.

13. The method of claim 12, wherein the expression vector is a viral vector, optionally wherein the viral vector is a lentiviral vector.

14. (canceled)

15. The method according to claim 1, wherein the intermediate cell type is a cell having an increased level of stem cell-like characteristics and/or epithelial characteristics compared to the first cell type.

16. The method according to claim 1, further comprising the step of detecting expression of integrins, pluripotent and/or stem cell markers to determine that said first cell type has transitioned to said intermediate cell type.

17. The method as claimed in claim 16, wherein an increased expression of integrin β4 and integrin β6 relative to the first cell type is indicative of an intermediate cell.

18. The method according to claim 1 further comprising contacting the intermediate cell type with a composition comprising a second agent to effect the reprogramming of said intermediate cell type to the second cell type.

19. The method according to claim 18, wherein said second agent ligates one or more integrin subunits selected from the group consisting of α1, α2, α3, α4, α5, α6, α7, α8, α9, α10, α11, αv, αD, αL, αM, αX, αE, αIIb, β1, β2, β3, β4, β5, β6, β7 and β8.

20. The method according to claim 19, wherein said second agent ligates an integrin subunit selected from the group consisting of α2, α3, α6, αv, α5, β1, β3, α4, α5, α6, and combinations thereof, optionally wherein the combination of subunits is selected from αvβ3, αvβ5, αvβ6, α6β1, α6β4, α2β1, α3β1 and α5β1.

21. (canceled)

22. The method according to claim 18, wherein said second agent comprises one or more ligands that binds to said at least one integrin to cause ligation of said integrin.

23. The method according to claim 22, wherein said one or more ligands comprises one or more extracellular matrix components, optionally wherein the extracellular matrix component is selected from a group consisting of laminin, collagen, entactin, and Geltrex.

24. The method according to claim 23, wherein the one or more extracellular matrix components are derived from basement membrane.

25. (canceled)

26. The method according to claim 18, wherein contacting the intermediate cell type with a composition comprising said second agent is conducted for a duration sufficient to effect reprogramming to the second cell type, optionally wherein the duration of contact is selected from at least about 12 hours, about 18 hours, about 24 hours, about 30 hours, about 36 hours, about 48 hours, about 2.5 days, about 3 days, about 3.5 days, about 4 days, about 4.5 days, about 5 days, about 5.5 days, about 6 days, about 6.5 days or about 7 days.

27. (canceled)

28. The method according to claim 1, wherein the method further comprises contacting said first cell type that has been contacted with said first agent to provide said intermediate cell of said second cell type and said second agent to effect the reprogramming of said intermediate cell type to the second cell type with an additional agent that is capable of effecting further reprogramming and/or modification of the growth and/or proliferative characteristics of the first cell type.

29. The method according to claim 28, wherein the additional agent is (a) a growth factor selected from the group consisting of fibroblast growth factor 2 (FGF2), epidermal growth factor (EGF), hepatocyte growth factor (HGF) and keratinocyte growth factor (KGF); and/or (b) a conditioned medium such as a conditioned medium from a rat insulinoma (Rin5f) cell line; and/or (c) a medium selected from B-27® supplement (comprising BSA, transferrin, insulin, progesterone, putrescine, sodium selenite, biotin, 1-carnitine, corticosterone, ethanolamine, d(+)-galactose, glutathione (reduced), linolenic acid, linoleic acid, retinyl acetate, selenium, t3 (triodo-1-thyronine), dl-α-tocopherol (vitamine e), dl-α-tocopherol acetate, catalase, superoxide dismutase) and TeSR™2 (comprising DMEM/F12 (liquid), L-ascorbic Acid, selenium, transferrin, NaHCO3, glutathione, L-glutamine, defined lipids, thiamine, β-mercaptoethanol, albumin, insulin, FGF2, TGFβ1, pipecolic acid, LiCl, GABA).

30. The method according to claim 28, comprising providing the additional agent for a duration sufficient to effect the further reprogramming to the second cell type and/or modification of the growth and/or proliferative characteristics of the cell, optionally wherein the duration is selected from the group consisting of at least about 1 day, about 2 days, about 3 days, about 4 days, about 5 days, about 6 day and about 7 days.

31. (canceled)

32. The method according to claim 1, further comprising providing a cellular support to support growth of the cells being reprogrammed.

33. The method according to claim 32, wherein said cellular support comprises a nanotopographical scaffold, optionally wherein said nanotopographical scaffold comprises uniform arrays of silica nanoparticles.

34. (canceled)

35. The method according to claim 1, wherein the first cell type is selected from the group consisting of mesenchymal cells, primary cancer cells, and cancer cell lines, optionally wherein the mesenchymal cell is a fibroblast cell.

36. (canceled)

37. The method according to claim 1, wherein the second cell type is selected from the group consisting of epithelial cells, cells with epithelial characteristics, neuronal cells, stem cells, cells with stem cell-like characteristics, and cancer stem cells.

38.-42. (canceled)

43. A method for treating a patient in need of cell-based therapy or tissue replacement, comprising administering to said patient a reprogrammed cell obtained according to the method of claim 1.

44. A method for treating a patient in need of cancer therapy, comprising delivering a bioactive to said patient using as a vehicle, a reprogrammed cell obtained according to the method of claim 1.

45. (canceled)

46. (canceled)


SEQUENCE LISTING (72 sequences)

SEQ ID NO: 1 - DNA, 20 bp, Artificial Sequence
  Primer
ttgacgctggggctggcatt

SEQ ID NO: 2 - DNA, 20 bp, Artificial Sequence
  Primer
gtgctcttgctggggctggt

SEQ ID NO: 3 - DNA, 22 bp, Artificial Sequence
  Primer
cctacaatgttggtctcccaga

SEQ ID NO: 4 - DNA, 23 bp, Artificial Sequence
  Primer
agtaaccagttgccttttggatt

SEQ ID NO: 5 - DNA, 21 bp, Artificial Sequence
  Primer
tcaacctggatacccgattcc

SEQ ID NO: 6 - DNA, 19 bp, Artificial Sequence
  Primer
gctctgtctgccgatggag

SEQ ID NO: 7 - DNA, 20 bp, Artificial Sequence
  Primer
tcgtgtccgctagtgcctcc

SEQ ID NO: 8 - DNA, 20 bp, Artificial Sequence
  Primer
gatgcaggccacagggttcc

SEQ ID NO: 9 - DNA, 19 bp, Artificial Sequence
  Primer
atgcacgcggatcgagttt

SEQ ID NO: 10 - DNA, 23 bp, Artificial Sequence
  Primer
ttcctgcttcgtattaacatgct

SEQ ID NO: 11 - DNA, 21 bp, Artificial Sequence
  Primer
atctgtgaggtcgaaacagga

SEQ ID NO: 12 - DNA, 23 bp, Artificial Sequence
  Primer
tggagcatactcaacagtctttg

SEQ ID NO: 13 - DNA, 22 bp, Artificial Sequence
  Primer
agcagaccaagttccggcagca

SEQ ID NO: 14 - DNA, 22 bp, Artificial Sequence
  Primer
gcgccatcagcactgtgtccac

SEQ ID NO: 15 - DNA, 19 bp, Artificial Sequence
  Primer
tctcggtgtgatctgaggg

SEQ ID NO: 16 - DNA, 19 bp, Artificial Sequence
  Primer
tggcgaacctgtagctgga

SEQ ID NO: 17 - DNA, 22 bp, Artificial Sequence
  Primer
gcgagagaagaagcaggcacat

SEQ ID NO: 18 - DNA, 20 bp, Artificial Sequence
  Primer
aaagagccgttcccgtggtg

SEQ ID NO: 19 - DNA, 20 bp, Artificial Sequence
  Primer
acgctgtgtcatccaacggg

SEQ ID NO: 20 - DNA, 22 bp, Artificial Sequence
  Primer
cctcctgggtgaattcgggctt

SEQ ID NO: 21 - DNA, 22 bp, Artificial Sequence
  Primer
ctctctgtggttatggctcagt

SEQ ID NO: 22 - DNA, 23 bp, Artificial Sequence
  Primer
ctgttggaatggtcactcgaaat

SEQ ID NO: 23 - DNA, 24 bp, Artificial Sequence
  Primer
ggacctgacagtaaatggggaaca

SEQ ID NO: 24 - DNA, 24 bp, Artificial Sequence
  Primer
acaactgctatcaccacaaccaca

SEQ ID NO: 25 - DNA, 21 bp, Artificial Sequence
  Primer
ggcatccagaacgagaaggag

SEQ ID NO: 26 - DNA, 23 bp, Artificial Sequence
  Primer
attgtccacagtatttgcgaaga

SEQ ID NO: 27 - DNA, 20 bp, Artificial Sequence
  Primer
cacctgccagcactcaagag

SEQ ID NO: 28 - DNA, 21 bp, Artificial Sequence
  Primer
agggatcctcagtgtcgtcaa

SEQ ID NO: 29 - DNA, 18 bp, Artificial Sequence
  Primer
cagcagcttgcggaaggt

SEQ ID NO: 30 - DNA, 26 bp, Artificial Sequence
  Primer
tgttttattctctcaaatccctcttg

SEQ ID NO: 31 - DNA, 19 bp, Artificial Sequence
  Primer
tctcggcttcagggagtca

SEQ ID NO: 32 - DNA, 23 bp, Artificial Sequence
  Primer
cgctttttgtttgatcctctttg

SEQ ID NO: 33 - DNA, 25 bp, Artificial Sequence
  Primer
tggagaaggagaagctggagcaaaa

SEQ ID NO: 34 - DNA, 24 bp, Artificial Sequence
  Primer
ggcagatggtcgtttggctgaata

SEQ ID NO: 35 - DNA, 20 bp, Artificial Sequence
  Primer
ctgagctggttgcctcatgt

SEQ ID NO: 36 - DNA, 20 bp, Artificial Sequence
  Primer
aaagcaaggcaagctttggg

SEQ ID NO: 37 - DNA, 20 bp, Artificial Sequence
  Primer
gcgggggaatggaccttgta

SEQ ID NO: 38 - DNA, 21 bp, Artificial Sequence
  Primer
ttcctgcaaagctcctaccgt

SEQ ID NO: 39 - DNA, 20 bp, Artificial Sequence
  Primer
cttccctccgcatcccgtca

SEQ ID NO: 40 - DNA, 21 bp, Artificial Sequence
  Primer
aaagccagcatgtcaccctcc

SEQ ID NO: 41 - DNA, 21 bp, Artificial Sequence
  Primer
acggacaagtacaggctgcac

SEQ ID NO: 42 - DNA, 22 bp, Artificial Sequence
  Primer
ccagaagggaagcgtgatgaca

SEQ ID NO: 43 - DNA, 21 bp, Artificial Sequence
  Primer
aaccttcctcagctatgcccg

SEQ ID NO: 44 - DNA, 20 bp, Artificial Sequence
  Primer
cagccgcaagaccccaaaga

SEQ ID NO: 45 - DNA, 19 bp, Artificial Sequence
  Primer
ccaaagcgaggcaccctta

SEQ ID NO: 46 - DNA, 21 bp, Artificial Sequence
  Primer
ggagagtaggctgccatgagg

SEQ ID NO: 47 - DNA, 24 bp, Artificial Sequence
  Primer
gagcatgcagaagcgcagatcaaa

SEQ ID NO: 48 - DNA, 24 bp, Artificial Sequence
  Primer
tatggctgatgctctggcagaagt

SEQ ID NO: 49 - DNA, 22 bp, Artificial Sequence
  Primer
ctgtggtgctgactgtcgtaga

SEQ ID NO: 50 - DNA, 20 bp, Artificial Sequence
  Primer
ggtagggcaggccaagggtc

SEQ ID NO: 51 - DNA, 20 bp, Artificial Sequence
  Primer
gaaagcgcaagtcctcaaag

SEQ ID NO: 52 - DNA, 20 bp, Artificial Sequence
  Primer
tgggtaggagatggagatgc

SEQ ID NO: 53 - DNA, 20 bp, Artificial Sequence
  Primer
ctgatgcttttgtgcgagaa

SEQ ID NO: 54 - DNA, 20 bp, Artificial Sequence
  Primer
acttggctcttcggttctga

SEQ ID NO: 55 - DNA, 20 bp, Artificial Sequence
  Primer
gggcgaccaagaacaggact

SEQ ID NO: 56 - DNA, 20 bp, Artificial Sequence
  Primer
gtgtgggactgcctggtgat

SEQ ID NO: 57 - DNA, 22 bp, Artificial Sequence
  Primer
ggtggaggagaagaagaccagg

SEQ ID NO: 58 - DNA, 20 bp, Artificial Sequence
  Primer
ggcatcaggctccacagtgt

SEQ ID NO: 59 - DNA, 21 bp, Artificial Sequence
  Primer
ccttgaacgcaaagtggaatc

SEQ ID NO: 60 - DNA, 23 bp, Artificial Sequence
  Primer
gacatgctgttcctgaatctgag

SEQ ID NO: 61 - DNA, 20 bp, Artificial Sequence
  Primer
tactggcctggaaccgggaa

SEQ ID NO: 62 - DNA, 20 bp, Artificial Sequence
  Primer
accagttggggaagctcgtc

SEQ ID NO: 63 - DNA, 20 bp, Artificial Sequence
  Primer
acggcctagcgagtggttct

SEQ ID NO: 64 - DNA, 20 bp, Artificial Sequence
  Primer
gattggggtcggagggcttc

SEQ ID NO: 65 - DNA, 20 bp, Artificial Sequence
  Primer
agctttcagacccccatgcc

SEQ ID NO: 66 - DNA, 22 bp, Artificial Sequence
  Primer
tggccagcccagaaaaagttga

SEQ ID NO: 67 - DNA, 19 bp, Artificial Sequence
  Primer
gggcagatcctgtccaagc

SEQ ID NO: 68 - DNA, 21 bp, Artificial Sequence
  Primer
gtgggtttccaccattagcac

SEQ ID NO: 69 - DNA, 21 bp, Artificial Sequence
  Primer
cgtgctatgaaggaagatgga

SEQ ID NO: 70 - DNA, 18 bp, Artificial Sequence
  Primer
tgcccagttcgtttcagt

SEQ ID NO: 71 - DNA, 20 bp, Artificial Sequence
  Primer
cggagcacggagacgggtat

SEQ ID NO: 72 - DNA, 20 bp, Artificial Sequence
  Primer
ttggaaccacgacgcccttg